(19) United States
(12) Patent Application Publication
(10) Pub. No.: US 2013/0304002 A1
(43) Pub. Date: Nov. 14, 2013

(54) SHEET AND LIQUID COMBINATION SYSTEMS FOR DERMAL DRUG DELIVERY

(71) Applicant: Jie Zhang, Salt Lake City, UT (US)

(72) Inventor: Jie Zhang, Salt Lake City, UT (US)

(21) Appl. No.: 13/884,585

(22) PCT Filed: Nov. 8, 2011
(86) PCT No.: PCT/US2011/059813

§ 371 Date: Aug. 2, 2013

Related U.S. Application Data
(60) Provisional application No. 61/411,728, filed on Nov. 9, 2010.

(51) Int. Cl. A61F 13/00

(52) U.S. Cl. CPC A61F 13/00063
USPC 604/307; 424/443; 514/535; 604/304; 514/627; 514/567

(57) ABSTRACT

A dermal drug delivery system is provided which comprises at least two components, for example, a sheet of a solid and flexible material, and a vehicle liquid comprising a solvent and optionally other ingredients. A drug, which can be unstable in said solvent but needs the solvent for being delivered into the skin, can be impregnated in the sheet. Other ingredients, such as agents for fastening the drug on the sheet can also be impregnated in the sheet. These two components may be stored separately and joined either shortly before or at the time of application. To use the system, the vehicle liquid may be applied either on the target skin area or on the sheet, and the sheet may then be applied on the target skin area so that the vehicle liquid is positioned between the sheet and the skin and brought into contact with the ingredients impregnated in the sheet. After the sheet and the vehicle liquid are combined in this way, the ingredients in the sheet and in the vehicle liquid are joined to form a combined formulation that is capable of delivering a drug through the skin at a desired rate. The sheet may have low enough permeability to the solvent or its vapor to control the time it takes for the solvent to evaporate across the sheet. When an appropriate local anesthetic agent, such as a tetracaine, is the drug, some embodiments of the system can have wide applications in anesthesia and pain control.

### RELATED APPLICATIONS

This application claims priority to U.S. Provisional Application No. 61/411,728 filed Nov. 9, 2010, entitled “SHEET AND LIQUID COMBINATION SYSTEMS FOR DERMAL DRUG DELIVERY,” which is incorporated herein by reference in its entirety.

### BACKGROUND

Dermal drug delivery systems (comprising drug formulations applied on the skin or mucosa) are widely used in treating medical conditions not only in the “surface tissues” such as skin or mucosa but also in deeper tissues such as musculoskeletal tissues. Some dermal drug delivery systems, such as fentanyl patches, even deliver drugs targeted for the central nervous system.

Almost all commercially available dermal drug delivery products are “one part” products, meaning the user does not need to combine or assemble two or more parts prior to or during the application. Such “one part” topical products include creams, ointments, patches, and sprayed-on formulations.

“One part” products, as opposed to multiple-part products, are convenient to use, but have limitations. If a drug has poor stability when exposed to a necessary excipient, incorporating the drug and the excipient into a “one part” product can make the drug unstable. For example, tetracaine base is subject to hydrolytic degradation when exposed to water, so a water-containing cream makes the tetracaine in it unstable and thus likely needs to be refrigerated to achieve the desired shelf life. In other circumstances, it may be desirable to have pre-determined differential onset times of effects for two active ingredients in the same product, but putting the two active ingredients in a “one part” product makes achieving the desired effect onset time differential difficult.

In summary, there are situations in which it is desirable to have a dermal drug delivery system with two or more components, such as a waterless solid sheet impregnated with a drug (and optionally with excipients) and a water-containing liquid solvent vehicle for dissolving the drug and delivering the drug via transdermal permeation, that are assembled or joined just before or during the application.

### DETAILED DESCRIPTION

In some embodiments of the current invention, the dermal drug delivery system comprises two components: the first component comprises a sheet of a solid and flexible material impregnated with certain ingredient(s); the second component comprises a vehicle liquid comprising a solvent. The second component may optionally further comprise other ingredients. These two components are stored separately and joined either shortly before or at the time of application. This system is generally referred to as a Sheet and Liquid Combination system hereafter. To use the system, the vehicle liquid is applied either on the target skin area or on the sheet, and the sheet is then applied on the target skin area so that the vehicle liquid is either between the sheet and the skin, absorbed into the solid sheet in whole or in part, or at least partially absorbed into the sheet and partially present between the sheet and the skin. After the sheet and the vehicle liquid are combined in this pre-designed way, the ingredients in the sheet and in the vehicle liquid are joined to form a combined (new) formulation that is capable of delivering the drug through the skin at the desired rates.

At least one of the two components comprises a drug (active ingredient). In some of the embodiments, each component alone is not able to deliver the drug at a desired rate, but the combination is. In other embodiments, one of the two components alone may be able to deliver the drug at a desired rate, but the other component provides another desirable property. For instance, a low viscosity drug solution may be able to deliver the drug into the skin at a desired rate if it is kept on the skin for long enough time. However, it is difficult to keep a low viscosity solution on skin for long time. Therefore, a system comprising a low viscosity drug solution in one component and a sheet with a liquid retention layer and a barrier film (discussed in further detail below) as another component may be used to keep the drug solution on the skin for long time.

It is noted that, as used in this specification and the appended claims, singular forms of “a,” “an,” and “the” include plural referents unless the content clearly dictates otherwise.

“Vehicle liquid”, or “liquid” in the Sheet and Liquid Combination system, means a liquid comprising a vehicle solvent system that is necessary to transdermally deliver the drug at rates high enough to achieve the desired effect(s). Vehicle liquid can be a free flowing liquid, a viscous liquid, liquid soaked in an absorbent sheet, a water-containing foam, or liquid in a solidified gel such as a hydrogel. Vehicle liquid may comprise only a single ingredient such as water, or multiple ingredients such as water, thickening agents, adhesion agents, etc. Vehicle liquid may have a color so that it is easier to see where it is spread on the skin or the sheet. In some of the embodiments of the current invention, the pH of the vehicle liquid can be important to the rate of delivery of the drug into the skin. In such embodiments, the pH of the vehicle liquid should be such that the vehicle liquid has sufficient solubility for the drug to facilitate the delivery of the drug at the desired rate, and (when the drug is an ionic substance) produces ratio(s) of unionized to ionized drug molecules in the vehicle liquid that can facilitate the permeation of the drug into human skin. In some embodiments of the current invention the pH of the vehicle liquid, initially or during the drug delivery process, needs to be such that a significant portion of the drug molecules dissolved in the vehicle liquid is in the unionized form, because for many drugs the unionized molecules can permeate normal human skin faster than the ionized ones. In some embodiments of the current invention, the drug to be delivered is a base and the pH of the vehicle liquid is not more than 1.5 pH units lower than the pKa of the drug, and may be not more than 1.0 unit lower than the pKa of the drug. In another embodiment, the vehicle liquid has an initial pH more than 1.0 pH unit lower than the pKa of the drug, but it has a weak pH buffer capacity so that when it is in contact with the sheet, the drug and/or a pH modifying agent impregnated in the sheet can dissolve into it and increase the pH to not more than 1.0 pH unit lower than the pKa of the drug. In another embodiment, the vehicle liquid (such as distilled water) has a weak pH buffer capacity. When such a vehicle liquid and the solid sheet are joined, the substance(s) in the solid sheet determines the pH of the liquid after the substance(s) in the solid sheet dissolves into the liquid. For example, the vehicle liquid can have a pH of about 7.0 but very weak pH buffer capacity (distilled water is such a vehicle liquid). When it is brought in contact with a sheet impregnated with a sufficient amount of tetracaine base per cm², some of the tetracaine (a base) dissolves into the vehicle liquid and increases its pH to 7.5 or higher. As a result, the pH of the vehicle liquid is increased sufficiently during the application period to facilitate fast permeation of the tetracaine into the skin. In some embodiments, the vehicle liquid is contained in a spray bottle and is sprayed onto the target skin area or the sheet prior to the application of the sheet on the target skin area. In other embodiments, the vehicle liquid is contained in a bottle with an applicator, such as, without limitation, a brush, on the lid, or separate from the lid, but capable of being attached to the lid, and is applied onto the target skin area or the sheet with the applicator prior to the application of the sheet on the target skin area. In other embodiments, the vehicle liquid is a viscous aqueous solution contained in a squeeze bottle with a long nozzle for dispensing and spreading the vehicle liquid on the skin or solid sheet. In some other embodiments, the vehicle liquid is essentially water (may contain color agent or preservative), so the pH of the vehicle liquid will be altered and determined by the substance(s) impregnated in the sheet after the vehicle liquid and the solid sheet are joined and the substance(s) gets dissolved in the liquid.

A “layer of vehicle liquid” means a continuous layer of the vehicle liquid, a substantially two dimensional presence but not necessarily continuous layer of the vehicle liquid (e.g., densely populated fine droplets of the vehicle liquid), or the vehicle liquid existing in a substantially two dimensional sheet of material (e.g. absorbed into a sheet of material to form a “wet” sheet).

“Sheet,” as used in describing the Sheet and Liquid Combination system of the current invention, means a sheet of solid material such as a paper, film, tape, fabric, sponge, or a combination thereof, that is impregnated with at least one of the ingredients necessary for the dermal drug delivery. The sheet can have a moisture vapor transfer rate (MVTR) that is very low, so that it keeps almost all of the water in the vehicle liquid placed between it and the skin for the entire duration of the application period. Alternatively, the sheet can have a pre-determined MVTR that allows water in the vehicle liquid to evaporate through it at rates such that sufficient amount of water in the vehicle liquid placed between it and the skin stays long enough to deliver the desired amount of the drug, but by the end of the application, enough of the water has evaporated so that there is minimal or no residue water left on the skin. This pre-determined MVTR is defined as “dry-ending MVTR”.

In some of the embodiments of the current invention, once the sheet and the vehicle liquid are joined and applied on the skin, the solvent in the vehicle liquid is present long enough to deliver a predetermined amount of the drug impregnated in the sheet into the skin. In such embodiments, the sheet's MVTR must be low enough to keep the solvent present long enough to deliver the predetermined amount of the drug.

“MVTR” means moisture vapor transfer rate, as measured with methods commonly used in the industry, such as those used by 3M Co. When a sheet is said to have a certain MVTR value, it means at least some part of the sheet has that MVTR value, and potentially that much or most of the sheet's area has that MVTR value, but it does not necessarily mean that the entire sheet area has that MVTR value.

One of the shortcomings of traditional dermal drug delivery patches is their finite sizes and shapes. For treating conditions with irregular and variable target skin areas, the fixed shape and size can be a problem. For example, the skin area suffering from pain associated with post herpetic neuralgia can have various shapes and sizes, so that covering it with a patch with fixed area and shape is difficult or impossible. To mitigate this problem, Lidoderm Patch (Endo Pharmaceutical) is often cut with scissors to fit the lesion area, which can be inconvenient to the user. To address this problem, in one of the embodiments of the current invention, the drug-impregnated sheet is in the shape of a roll and may have a pattern of perforation or partial-cut lines that allow the user to use just hands to easily tear a piece of the sheet with the shape and size that approximately fits the target skin area. Alternatively, the sheet is not in a roll but in the form of a large sheet with a pattern of perforation or partial-cut lines, for the same purpose. Of course, the sheet can also simply be a large sheet, and the user can cut it into the size and shape desired to fit the application area.

In some embodiments of the current invention, the sheet has a “lateral diffusion function” or comprises a “lateral diffusion layer”. The lateral diffusion layer is typically a layer of material very absorbent to the vehicle liquid, such as water. When a droplet of the vehicle liquid is placed on the lateral diffusion layer, it is quickly absorbed into the layer and spread laterally to cover an area much larger than the initial size, as measured by cross-sectional area, of the droplet. (When a drop of water is placed on a Kleenex tissue, it will quickly spread laterally into a circle whose diameter is much larger than the diameter of the initial water drop. However, a drop of water placed on an aluminum foil will not diffuse much laterally. In this comparison, the Kleenex tissue has the lateral diffusion function and it or a material with similar lateral diffusion function may be used as a lateral diffusion layer in a sheet, while the aluminum foil does not have the lateral diffusion function and cannot be used as a lateral diffusion layer in a sheet). The lateral diffusion layer is typically the layer in the sheet that is in contact with the vehicle liquid when the combination of the sheet and the vehicle liquid is applied on the skin. The lateral diffusion layer can make sure that the vehicle liquid can evenly spread over the desired area under the sheet, even if the initial application of the vehicle liquid on the skin or the sheet is not very even. For example, water as the vehicle liquid can be sprayed on the skin to cover the target skin area with densely populated water beads, but not quite a continuous layer of water. The lateral diffusion layer applied over the water beads will quickly absorb the water beads. The absorbed water will then quickly spread laterally, so that the entire sheet area will have even water distribution. Many absorbent materials may be used as the material for the lateral diffusion layer, including gauze (woven or non-woven), paper, foam (especially open-cell foam), cloth, and other fabric materials. For example, the fabric (gauze) layer in the fabric-tape laminate sheets in many of the following Examples is the lateral diffusion layer.

Unless specified otherwise, when a tape-fabric or film-fabric laminate sheet is said to be “applied to the skin,” it means the sheet is applied in such a way that the fabric side of the laminate is the side that is in direct contact with the skin and the applied vehicle liquid.

A lateral diffusion layer can also be useful in the manufacturing process. It is generally much easier to dispense precise volumes of solution on a sheet of material than cast a thin layer of solution with precise thickness on a sheet of material. If the droplets of the solution containing the drug (and optionally the fastening agent) are dispensed on a lateral diffusion layer (in this case the lateral diffusion layer is very absorbent to the solution containing the drug and fastening agent), the solution is quickly absorbed into the layer, spread laterally, and reach even distribution within the layer within minutes or even seconds. As a result, the drug and the fastening agent dissolved in the solution also reach even distribution within the lateral diffusion layer. The volatile solvent in the lateral diffusion layer is then evaporated off, leaving evenly distributed drug and fastening agent in the lateral diffusion layer (and thus the sheet). For example, the gauze layer in the gauze-tape laminate sheet in Examples 3-5, among others, functioned as the lateral diffusion layer for the manufacturing of the sheet impregnated with the drug and fastening agent.

Since the lateral diffusion layer is absorbent to the vehicle liquid, it typically has very high MVTR (moisture vapor transfer rate). Therefore, in order to maintain water in the vehicle liquid between the sheet and skin for sufficient time to deliver the desired amount of the drug into the skin, the lateral diffusion layer is often laminated with a “MVTR control layer” to form a sheet that has both the lateral diffusion function and proper MVTR. Typically, the “MVTR control layer” is a layer of material that has much lower MVTR than that of the lateral diffusion layer and thus dominantly determines the overall MVTR for the sheet. The MVTR control layer is typically a layer of plastic film or tape with desired MVTR. For example, the 3M 9832 polyurethane tape in Example 4 and 5 is the MVTR control layer in the laminate sheet.

The MVTR control layer can also be or comprise a barrier film with adequate MVTR. In the current invention, the phrase “barrier film” means a film with MVTR lower than 5,000 g/m²/24 hour, and in some instances lower than 2,000 g/m²/24 hour. It should be noted that many tapes used in the current invention, such as 3M 9832, 3M 9834 tapes, comprise a barrier film layer. A tape is typically a film coated with a layer of adhesive.

The fabric (lateral diffusion) layer and the MVTR control layer can be conveniently laminated by using the layer of adhesive coated on MVTR control layer, if the MVTR control layer is a tape with adhesive coating on one side. For example, the 3M9832 tape is a polyurethane film with one side coated with a layer of adhesive. As shown in some of the examples below, a layer of fabric can be placed on the adhesive side of the tape to form a fabric/barrier film laminate. This adhesive lamination process is relatively easy to complete, and use of medical-grade tape should helps to assure safety in human use. However, the inventor surprisingly found evidence suggesting that some adhesives used for lamination (lamination adhesives) interact with the tetracaine formulation in the sheet (chemically or physically) so that the anesthetizing ability of the sheet could be compromised after long term storage. Further, the inventor determined that one of the lamination adhesives tested has much less or no tendency to compromise the anesthetizing ability. To avoid the potential problem of adverse interaction between the lamination adhesive and the drug formulation, in some of the embodiments of the current invention, the fabric is laminated to the barrier film (MVTR control layer) by heat.

It should be noted that for any given pair of barrier films and fabric materials, there may or may not exist a window of heating temperature and duration in a heat lamination process that may be used to successfully laminate the two materials without damaging the barrier film.

Through experimentation, the inventor found a window of heating temperature/duration that allows a rayon polyester fabric (a preferred fabric for many embodiments of the current invention) and a polyurethane film (a preferred MVTR control layer material) to be securely laminated together with heat without damaging either the fabric or the film.

“Fabric layer” or “fabric” means a material or a layer of material that is absorbent of water or water based solution, including woven and non-woven materials. For example, a layer of non-woven rayon-polyester blend material as that used in some of the examples below is a fabric layer. In contrast, wax-coated paper is not “fabric” by the definition herein because it is not absorbent to water.

In some of the embodiments, the sheet in the sheet and liquid combination system can have a liquid retention layer for keeping the vehicle liquid on the skin for long enough time to deliver the desired amount of the drug. As can be seen in some of the examples below, maintaining water on the skin for long enough time can be very important in delivering sufficient amount of drug into the skin to achieve desired clinical effect. A liquid retention layer, such as a fabric layer in a fabric-barrier film laminate sheet, can absorb the vehicle fluid and keep it relatively evenly available to the skin for the desired length of time. A liquid retention layer can have the lateral diffusion function and can be the lateral diffusion layer at the same time.

In some embodiments, the drug is incorporated into the MVTR control layer itself, and the lateral diffusion layer is not used. For example, tetracaine can be incorporated into a barrier film which itself is the MVTR control layer. The means of incorporating tetracaine into barrier film includes, without limitation, diffusing tetracaine into the barrier film and blending tetracaine into the monomers to be polymerized into the barrier film. For example, in Example 38, tetracaine was incorporated into the polyurethane film by diffusion. When this tetracaine-impregnated barrier film is used with a viscous aqueous solution (vehicle liquid), enough tetracaine was delivered into the skin to produce deep skin anesthesia. Drugs other than tetracaine, such as anti-infection agents, may also be incorporated into the MVTR control layer with similar methods. In those embodiments, the drug impregnated barrier film constitutes the sheet in the sheet and liquid combination system of the current invention.

“Fastening agent” means a substance that “fastens” a drug or an excipient on the sheet. Without the fastening agent, the drug or the excipient impregnated in the sheet may be only loosely held by the sheet and can be unintentionally removed from the sheet when the sheet is shaken, bent, touched, or rubbed. Substances that can bind with both the sheet and the drug or excipient can function as fastening agents. Such substances include but are not limited to polyvinylpyrrolidone (PVP), poly vinyl alcohol (PVA), ethyl cellulose, hydroxy propyl cellulose, carrageenan, and gum Arabic. However, in some embodiments, the drug is incorporated into the barrier film polymer itself (see Example 38). In those embodiments, a fastening agent may not be necessary.

“Adhesion agent” is a substance capable of facilitating the adhesion between the skin and the sheet. It can initially exist in the vehicle liquid. It can also initially exist in the sheet, and dissolve into the vehicle liquid when the vehicle liquid and the sheet are brought into contact. Substances that are soluble in the vehicle liquid and increase the vehicle liquid's adhesion to skin or to the sheet can be used as adhesion agents. Such substances include, but are not limited to, PVP, PVA, poly acrylic polymers such as the Carbomer polymers marketed by Noveon (e.g Carbopol 981), xantham gum, and gum Arabic. Adhesion agent can also be a combination of two or more substances. For example, polyvinylpyrrolidone-glycerin mixture and polyvinylpyrrolidone-poly ethylene glycol 400 mixture, with appropriate polyvinylpyrrolidone percentages, can be used as adhesion agents.

“Normal human skin” means human skin with an intact stratum corneum layer and normal skin temperature (typically in the range of 30-36° C.). Normal human skin can include skin that is suffering from a disease or pain but has an intact stratum corneum layer.

“Normal ambient conditions” means temperatures in the range of 20-35° C. and relative humidity in the range of 0 to 80%.

The phrases “anesthesia in skin”, “anesthetized skin”, “numbness”, and the like, mean the skin is anesthetized at least to the extent that it feels obviously numb when it is scratched or poked with the end of a straightened paper clip. This skin numbing effect can be observed in a single human subject (such as in some of the Examples) or can be observed in a formal clinical trial. Since there are often outliers in human testing, this term is used to denote that in a formal clinical trial, at least 70% of the subjects in a group of at least 24 subjects have the effect. Alternatively, the definition can be that the effect is statistically significant according to the US Food and Drug Administration's definition at the time of testing.

The term “free of water” when used to describe an environment or medium (such as the aforementioned solid sheets) in which exists tetracaine or other drugs that are subject to hydrolytic degradation, means that the environment does not have a sufficient amount or concentration of water to cause the tetracaine or said other drugs to lose more than 2% per year to hydrolytic degradation at room temperature.

The term “subject to hydrolytic degradation” means that a drug, in a formulation containing a concentration of water which is sufficient to deliver the drug at sufficient rates to achieve the desired clinical effect, is subject to a hydrolytic degradation process with high enough rates so that a shelf life of at least one year at room temperature (by US FDA methods and definition) cannot be obtained.

The term “appropriate quantity” when referring to the quantity of the vehicle liquid applied on the skin or sheet means a quantity of the vehicle liquid that is high enough and can last long enough to allow a sufficient amount of the drug to be delivered transdermally into the skin to achieve the desired clinical effect(s), but not so high as to cause problems such as overflow or running. The “appropriate quantity” can depend on the MVTR of the particular sheet, among other factors, and can be in the range of 2 to 200 milligrams per cm² (mg/cm²), including the range of 10 to 50 mg/cm², and including the range of 20 to 30 mg/cm².

The phrase “between the skin and the sheet”, and the like, when referring to the position of the vehicle liquid relative to the skin and the sheet means the vehicle liquid is between the skin and the sheet and includes situations in which the vehicle liquid is applied onto or absorbed into the sheet, or partially absorbed into the sheet and partially present between the sheet and the skin, and the sheet is applied on the skin.

“Target skin area” in general means an area of human (or other mammal) skin into which the delivery of the drug is expected to produce the desired clinical effect(s). For anesthetizing the skin before painful procedures, reducing the pain associated with shingles, and other pain associated with diseases or trauma of the skin, the target skin area can be the skin area suffering from the pain. For musculoskeletal pain, the target skin area can be the skin area under or adjacent to which the musculoskeletal pain exists. Target skin area can also be the skin area over a “trigger point”, a hyperirritable spot in the tissue (usually muscle tissue) that sometimes can cause pain quite distant from the trigger point itself. Target skin area can also be a skin area over a tissue into which physicians would inject a local anesthetic or other drugs to reduce pain (e.g., skin area over the site into which physicians inject lidocaine to reduce shoulder pain). Target skin area can be an area of diseased or normal skin.

The term “properly adhered”, “proper adhesion”, and the like, when referring to the adhesion of the sheet on human skin, means that the adhesion is such that the sheet can stay on a normal human skin area under normal ambient conditions for at least 15 minutes no matter how the skin area is positioned (e.g., face up, face down, or at an angle such that the sheet is vertical to the ground).

“Rubbing alcohol” in the Examples means Western Family brand rubbing alcohol which contains 70% isopropyl alcohol by volume.

“Tetracaine” can mean tetracaine base or a salt of tetracaine (e.g. tetracaine hydrochloride). Similarly, any drug listed in this disclosure includes its salt(s).

“Pain reduction” can mean the reduction of pain sensed by a human being in general. Pain reduction can also mean statistically significant reduction of pain as measured by methods commonly used in clinical trials employing commonly used patient selection criteria and test conditions. Such methods include, without limitation, the visual analog pain scale method.

Many drugs can be delivered using the systems and methods of the current invention. These drugs include, but are not limited to, local anesthetics such as lidocaine, tetracaine, prilocaine, bupivacaine, benzocaine, ropivacaine, etidocaine, mepivacaine, dibucaine; non-steroidal anti-inflammatory drugs (NSAIDs) such as diclofenac and ketoprofen; capsaicin; drugs that are used to treat neuropathic pain such as N-methyl-D-asparate (NMDA) receptor antagonists (e.g. gabapentin) and ketamine. In some systems of the current invention, the drugs which particularly benefit from the systems and methods of the current invention are those which are subject to hydrolytic degradation when in contact with water.

In some embodiments of the current invention, a system for delivering a local anesthetic agent into human skin comprises a first component and a second component. The first component comprises a sheet of a solid and flexible material and is impregnated with a local anesthetic agent. If the local anesthetic agent is an ester type local anesthetic agent such as tetracaine or benzocaine, the sheet is free of water because these local anesthetics are subject to hydrolytic degradation. The sheet can also be free of water even if the local anesthetic is the amide type which is not subject to significant hydrolytic degradation. But for ester types, being free of water is more important. The second component comprises a vehicle liquid comprising water. To use the system to deliver the local anesthetic into human skin or skin of another mammal, the vehicle liquid is applied either on the target skin area or on the sheet, and the sheet is then applied on the target skin area, so that the vehicle liquid is between the sheet and the skin. Once the vehicle liquid is in contact with the sheet and the skin, the local anesthetic impregnated in the sheet begins to dissolve into the vehicle liquid and is delivered into the skin via the vehicle liquid. The sheet is constructed to have a low enough MVTR to keep the water in the vehicle liquid between the sheet and the skin for a time sufficient to deliver the needed amount of drug. The system is maintained in place for a time sufficient, for example 30 minutes, to deliver a sufficient amount of the local anesthetic agent to anesthetize the skin or to achieve a certain analgesic or anesthetic effect. An adhesion agent can be incorporated in the vehicle liquid to enable the vehicle liquid to serve as a weak “glue” to keep the sheet properly adhered on the skin for the duration of the application period. The adhesion can be strong enough so that the sheet stays on the skin even if the patient moves or changes the position of the body part that contains the skin area. Alternatively, the adhesion agent can be impregnated in the sheet, and dissolves into the vehicle liquid when vehicle liquid is brought into contact with the sheet. The vehicle liquid can be applied on the target skin area or the sheet in several different ways, including sprayed on the skin or the sheet, brushed on the skin or the sheet; soaked in a sheet of absorbent material (to form a “wet sheet”) which is then applied on the skin or the sheet, or crosslinked into a solidified hydrogel sheet which is then applied on the skin or the sheet. Optionally, the sheet can have a dry-ending MVTR so that it can keep the water in the vehicle liquid under the sheet long enough to deliver a sufficient amount of the drug into the skin to achieve a desired anesthetic or analgesic effect, but by the end of the application period a sufficient amount of water in the vehicle liquid under the sheet has evaporated through the sheet so that when the sheet is removed from the skin at the end of the application period, the skin is substantially free of liquid so that the need to wipe off the residual liquid on the skin is avoided.

The dry-ending MVTR can be achieved in several ways: (1) Selecting a film or tape such as a MVTR control layer or barrier film with the appropriate MVTR and laminating a porous (very high MVTR) fabric onto it. The film or tape determines the MVTR for the overall sheet, and the drug or excipients can be impregnated into the fabric layer. For example, a polyurethane film or tape or a microporous film or tape with an appropriate MVTR can be used for this purpose. (2) Using a film that has the proper MVTR and is capable of accommodating the drug (optionally with the help of a fastening agent). A polyurethane film, microporous polyethylene or rayon film or tape may be used for this purpose. Similarly, a layer of foam sponge with the proper MVTR may also be used in this approach. (3) Dispensing a solution comprising a film-forming substance onto a layer of fabric material with very high MVTR, evaporate off the solvent and form a film on the fabric. If the film-forming substance and its quantity dispensed on the fabric sheet are selected properly, a film with the MVTR in the desired range can be formed. Optionally, the film-forming substance is not readily soluble in the vehicle liquid so that it is not easily destroyed during the application. In some embodiments of the current invention, the film-forming substance can also play the role of fastening agent. The ideal value of dry-ending MVTR for a particular system is dependent on factors such as the quantity and the composition of the vehicle liquid placed between the sheet and the skin, the length of the designed drug delivery period, and the conditions of the skin. Therefore, different systems with different applications can require different dry-ending MVTRs. In some embodiments, the dry-ending MVTR of the sheet is in the range of 100 to 10,000 gram/m²/24 hours. In some other embodiments, ending-dry MVTR of the sheet is in the range of 200 to 6,000 gram/m²/24 hours.

In some embodiments of the current invention, the vehicle liquid comprises a crosslinkable but uncrosslinked polymer and the sheet comprises a crosslinking agent capable of crosslinking the crosslinkable polymer in said vehicle liquid. When the vehicle liquid and the sheet are brought into contact, the crosslinking agent in the sheet diffuses into the vehicle liquid and crosslinks the polymer, which solidifies the vehicle liquid. When the sheet is removed from the skin after the application period, the solidified vehicle liquid, which is adhered to the sheet, is lifted with the sheet. No or very minimal residue is left on the skin.

The vehicle liquid can have an appropriate viscosity for facilitating the application on the skin or the sheet and the delivery of the drug into the skin. For vehicle liquid that is spread on the skin with a brush or a spatula or a Q-tip, if the viscosity is too low, maintaining an appropriate quantity of the vehicle liquid on the skin before the sheet is applied can be difficult because a low viscosity liquid can flow away from the target skin area easily. If the viscosity is too high, the application of it on the target skin area can be difficult. The vehicle liquid that is to be spread on the skin (as opposed to spayed on the skin) can have a viscosity in the range of 100 to 1 million centipoises, alternatively in the range of 500 to 200,000 centipoise, and alternatively in the range of 1,500 to 50,000 centipoise. However, in systems in which the vehicle liquid is to be sprayed on the sheet or skin, the viscosity of the vehicle liquid is preferably lower, and can be as low as that of water or lower.

The sheet can be optionally stretchable (elastic) so that it can better maintain intimate contact with the skin area if the skin area is stretched when the patient moves. It is desirable that the sheet can be stretched to increase its length by at least 5% in at least one direction without breaking.

The drug can be impregnated in the sheet with the help of a “fastening agent”. For example, tetracaine and poly vinyl alcohol (PVA) can be dissolved in an isopropyl alcohol:water solution (e.g. 50:50 by weight), which is then evenly dispensed into the absorbent fabric part of a sheet. After evaporating off the isopropyl alcohol and water, the PVA solid binds with both the fabric and the tetracaine, thus “fastens” the tetracaine on the sheet.

In another embodiment, a system for delivering tetracaine into human (or other mammalian) skin comprises a first component and a second component. The first component comprises a sheet of a solid and flexible material and is impregnated with tetracaine base, and optionally a fastening agent or optionally an adhesion agent. A single substance may serve as both the fastening agent and the adhesion agent. The sheet is free of water so the tetracaine impregnated in it is not subject to significant hydrolytic degradation before it is brought into contact with the vehicle liquid. The second component is a vehicle liquid comprising water and optionally an adhesion agent. The two components are stored separately. To use the system to deliver the tetracaine into human (or other mammalian) skin, the vehicle liquid is applied either on the target skin area or on the sheet, and the sheet is then applied on the target skin area, so that the vehicle liquid is between the sheet and the skin. Once the vehicle liquid is in contact with the sheet and the skin, tetracaine impregnated in the sheet begins to dissolve into the vehicle liquid and is delivered into the skin via the vehicle liquid. The sheet has a sufficiently low MVTR to keep an appropriate amount of the vehicle liquid between the sheet and the skin for a period of time sufficient to deliver an amount of tetracaine to achieve the desired anesthetic or analgesic effect (this MVTR is referred to as “sufficiently low MVTR for tetracaine delivery”). Sufficiently low MVTR for tetracaine delivery can depend on factors such as quantity of the vehicle liquid applied between the skin and the sheet as well as skin and ambient temperatures, and can mean MVTR values lower than 5,000 g/m²/24 hours. In certain embodiments, the MVTR of the sheet is in the range of 200 to 10,000 g/m²/24 hours. In other embodiments, the MVTR of the sheet is in the range of 600 to 6,000 g/m²/24 hours. The sheet has an MVTR control layer which is typically a barrier film or tape.

Optionally, the sheet also has the later diffusion function or has a later diffusion layer. The sheet can be a laminated sheet comprising a fabric layer and a barrier film, laminated together with adhesive or with heat. In its application, the system is kept on the skin for sufficient time to deliver a sufficient amount of tetracaine for the desired application. In some of these embodiments, the amount of tetracaine in each cm² of the sheet can be at least 0.1 mg. In others, the amount of tetracaine in each cm² of the sheet can be at least 0.15 mg or at least 0.3 mg. In other such embodiments, the amount of tetracaine in each cm² of the sheet can be between 0.5 mg and 3 mg, or between 1 and 2 mg. Tetracaine quantities higher than 3 mg/cm² will also work, but may be unnecessary. Therefore, in some of the embodiments, the average tetracaine quantity per unit sheet area (total tetracaine quantity divided by area of the sheet) is no more than about 3 mg/cm². The desired length of the application time can be dependent on the application. For example, with a properly made and used system (i.e. appropriate amount of tetracaine per cm², low enough sheet MVTR so that the skin under the sheet is kept wet for at least 30 minutes, appropriately formulated vehicle liquid, and an appropriate quantity of the vehicle liquid between the skin and the sheet), such as that in Example 1, normal human skin in normal ambient conditions can be anesthetized within 240 minutes, within 120 minutes, within 60 minutes, or even within 45 minutes. In these embodiments, tetracaine is not subject to significant hydrolytic degradation during storage since the sheet is free of water, thus providing a long shelf life. When tetracaine dissolves into the vehicle liquid after the sheet and the vehicle liquid are brought into contact with each other, the tetracaine becomes subject to hydrolytic degradation. However, since the application time is usually not longer than a few hours, tetracaine loss due to the hydrolytic degradation during the application period is minimal and is of no practical significance. The amount of the vehicle liquid applied between the skin and the sheet is preferably approximated to be in the range of from about 2 to about 200 mg/cm², or in the range of from about 10 to about 50 mg/cm².

Tetracaine in solutions with a low pH, such as 6.0 or lower, including 5.5 or lower, can have slow enough hydrolytic degradation rates to have a decent shelf life (e.g. 1-2 years) in room temperature storage. However, the pH of the vehicle solution has to be higher than about 6.5, including higher than about 7.5, to deliver tetracaine into intact human skin to achieve skin anesthesia within 60 minutes. For avoiding these conflicting pH requirements for stability and high delivery rates, a system in another embodiment of the current invention for delivering tetracaine into human skin comprises a first component and a second component. The first component comprises a sheet of a solid and flexible material and is impregnated with a pH modifying agent which is capable of increasing the pH of the vehicle solution when dissolved in it. The second component is a vehicle liquid comprising water, tetracaine, and having a pH lower than about 6.0, and the tetracaine degradation loss in the vehicle solution is less than 8% per year at room temperature, preferably less than 4% per year at room temperature. To use the system to deliver tetracaine into human skin, the vehicle liquid containing tetracaine is applied either on the target skin area or on the sheet, and the sheet is then applied on the target skin area so that the vehicle liquid is between the sheet and the skin. After the vehicle liquid is in contact with the sheet and the skin, the pH modifying agent impregnated in the sheet dissolves into the vehicle liquid and increases the pH of the vehicle liquid to higher than about 6.5, including higher than about 7.5. This pH increase converts many tetracaine molecules from the ionized state (associated with low skin permeability) to unionized state (associated with higher skin permeability), thus increasing the tetracaine delivery rates into the skin. The system is kept on the skin for sufficient time to deliver a sufficient amount of tetracaine for the desired application. The concentration of tetracaine in the vehicle solution, optionally in the form of tetracaine hydrochloride, can be from about 0.1% to about 20%, and, in certain embodiments, from about 0.4% to about 6%. Many bases or buffers can be used as the pH modifying agent, including sodium bicarbonate, phosphate buffer, and sodium borate.

In another embodiment, a system for delivering tetracaine into human skin comprises a first component and a second component. The first component comprises a sheet of a solid and flexible material and is impregnated with a tetracaine salt, such as tetracaine hydrochloride, and optionally a fastening agent or optionally an adhesion agent. The second component is a vehicle liquid comprising water and pH-modifying agent (the function of which is described in detail below), and optionally an adhesion agent. To use the system to deliver the tetracaine into human skin, the vehicle liquid is applied either on the target skin area or to the sheet, and the sheet is then applied on the target skin area, so that the vehicle liquid is between the sheet and the skin. Once the vehicle liquid is in contact with the sheet and the skin, the tetracaine salt impregnated in the sheet begins to dissolve into the vehicle liquid. The pH modifying agent in the vehicle liquid coverts many of the dissolved tetracaine molecules from the ionized species to unionized species which permeate into the skin at a higher rate since the unionized species has better skin permeability than the ionized species. The sheet is selected to have an MVTR such that the vehicle liquid applied between the sheet and the skin is kept there for a long enough time to deliver a sufficient amount of tetracaine to achieve the desired effect. The sheet has an MVTR layer which is typically a barrier film or tape. Optionally, the sheet also has a lateral diffusion function or has a lateral diffusion layer. The system is kept on the skin for sufficient time to deliver a sufficient amount of tetracaine for the desired application.

In some of these embodiments, the amount of tetracaine salt in each cm² of the sheet can be at least 0.1 mg. In others, the amount of tetracaine in each cm² of the sheet can be at least 0.15 mg or at least 0.3 mg. In yet others, the amount of tetracaine salt in each cm² of the sheet can be between about 0.5 mg and about 3 mg, or between about 1 to about 2 mg. The desired length of the application time can be dependent on the application. For example, with a properly made system (i.e. containing an appropriate amount of tetracaine salt per cm² and having a low enough sheet MVTR such that the skin under the sheet is kept wet for at least 30 minutes, and using an appropriately formulated vehicle liquid, with an appropriate quantity of the vehicle liquid between the skin and the sheet, on normal human skin in normal ambient conditions, can be anesthetized within 240 minutes, within 120 minutes, within 60 minutes, or even within 45 minutes.

Other embodiments of the current invention are related to a system comprising water and an MVTR control layer such as a barrier film (a film whose MVTR is lower than 5,000/m²/24 hour) and tetracaine, wherein said tetracaine is distributed co-extensively with said barrier film (either in the barrier film or in another layer of material co-extensive with the barrier film), and wherein said water is brought into contact with said tetracaine within one hour of application of said system on a mammal's skin.

In another embodiment, methods of using some of the aforementioned two-component drug delivery systems for producing anesthesia in human or other mammalian skin, including tissues under the skin, prior to painful procedures are provided. As described previously, in some of the embodiments, the first component of the system comprises a sheet of a solid and flexible material, and the second component comprises a vehicle liquid. A local anesthetic (such as tetracaine) is impregnated in the sheet, and the vehicle liquid comprises water. A fastening agent can be optionally impregnated in the sheet to fasten the tetracaine (or other local anesthetic) to the sheet. An adhesion agent can be impregnated in the sheet or incorporated in the vehicle liquid. The sheet and the vehicle liquid are stored separately. To use the system for producing the anesthesia, the vehicle liquid is applied either on the target skin area or on the sheet, and the sheet is then applied on the target skin area, so that the vehicle liquid is between the sheet and the skin. Once the vehicle liquid is in contact with the sheet and the skin, the local anesthetic impregnated in the sheet begins to dissolve into the vehicle liquid and is delivered into the skin via the vehicle liquid. The system is kept on the skin for sufficient time to deliver a sufficient amount of the local anesthetic agent to produce adequate anesthesia in the tissues. The “sufficient time” depends on factors such as the composition of the vehicle liquid, the MVTR of the sheet, the individual's skin permeability to the local anesthetic agent, the depth of the tissue to be anesthetized, and how painful the procedure is (if there is no anesthesia). The “sufficient time” can be as short as 15 minutes, especially for human facial skin or mucosa, but may be 30, 45, or 60 minutes. In situations where the procedure is particularly painful, such as laser tattoo removal or some biopsy procedures, the sufficient time may be as long as 120 minutes. It is also possible that the skin is not anesthetized after a relatively short application time, such as 15 to 30 minutes, but becomes anesthetized some time (e.g. 30 minutes) after the system is removed from the skin (see some Examples below). That is because some quantities of tetracaine can be stored in or under the stratum corneum layer during the application period and continue to penetrate deeper after the application period. Examples of painful procedures include, but are not limited to, needle injections; laser procedures such as laser tattoo removal, laser spider vein removal, laser hair removal, laser skin resurfacing; and the application of capsaicin-containing formulations on skin, botox or filler injections.

In another embodiment, a method of using some of the aforementioned two-component drug delivery systems for reducing the pain associated with herpes zoster in the pre-eruptive phase (preherpetic neuralgia), acute eruptive phase, or chronic phase (postherpetic neuralgia), is provided. As described previously, in some of the embodiments, the first component of the system comprises a sheet of a solid and flexible material, and the second component comprises a vehicle liquid. A local anesthetic (such as tetracaine) is impregnated in the sheet. The vehicle liquid comprises water. A fastening agent can be optionally impregnated in the sheet to fasten the tetracaine (or other local anesthetic) to the sheet. An adhesion agent can be impregnated in the sheet or incorporated in the vehicle liquid. The sheet and the vehicle liquid are stored separately. To use the system, the vehicle liquid is applied either on the skin area suffering from the pain or on the sheet, and the sheet is then applied on the skin area suffering from the pain, so that the vehicle liquid is between the sheet and the skin. When the vehicle liquid is in contact with the sheet and the skin, the local anesthetic impregnated in the sheet dissolves into the vehicle liquid and is delivered into the skin via the vehicle liquid. The system is kept on the skin for sufficient time to significantly reduce the pain. With an appropriately formulated system and method of using it, the pain can be significantly reduced within 60 minutes of the application. If the herpes zoster is in its acute (eruptive) phase and the stratum corneum layer of the skin (the main barrier of the skin) is broken, pain reduction can be achieved even sooner. If the skin is broken, has blisters or rash (significantly compromised or missing the stratum corneum) in the acute eruptive phase of a herpes zoster infection, the time to achieve significant pain reduction can be much shorter than if the skin has intact stratum corneum. In those cases it may be possible to achieve significant pain reduction within a few minutes following the application of the system. However, maintaining the system on the lesion for longer time, such as between 5 and 60 minutes, can be more beneficial because that allows more tetracaine to be delivered into the tissues under the sheet, especially fatty tissues that can store tetracaine, and result in a longer lasting pain reduction effect. If the skin is broken and sufficient amount of bodily fluid is oozing out of the lesion, the sheet may be applied directly to the lesion without the vehicle fluid, as the bodily fluid may work as the vehicle liquid. Tetracaine is an exemplary drug in this embodiment because it produces a longer analgesic effect after the drug formulation is removed from the skin than other commonly used anesthetics such as lidocaine and prilocaine. This long “tail” of analgesic effect after the drug delivery system is removed from the skin is particularly desirable for treating herpes zoster in its acute eruptive phase, because after applying the system on the diseased skin for as short as one hour, the skin can continue to enjoy pain reduction for many hours. This allows the skin to be treated by other topical medications. When tetracaine is used as the drug, the system is also desirable for reducing pain associated with herpes zoster in the chronic phase, also known as post herpetic neuralgia. That is because an application of the system for as short a time as one hour can produce significant pain reduction for many hours, for example 5-12 hours. The skin area is thus not covered with the treatment formulation or structure for most of the day, which minimizes occlusion-induced skin irritation and discomfort or interference with the patient's daily activities such as exercise, work, shower, and sleep. For example, the patient can apply the system for one hour every 6-12 hours and get significant pain reduction or even elimination around the clock. Because pain associated with post herpetic neuralgia can last months to years, the short application time coupled with long lasting pain reduction associated with a system comprising tetracaine can mean a significantly reduced skin irritation and discomfort, which may in turn provide a better quality of life for patients, when compared with other treatment options.

In another embodiment, a method of using some of the aforementioned two-component drug delivery systems for reducing neuropathic pain is provided. As described previously, in some of the embodiments, the first component of the system comprises a sheet of a solid and flexible material, and the second component comprises a vehicle liquid. A local anesthetic (such as tetracaine) is impregnated in the sheet, and the vehicle liquid comprises water. The sheet may also comprise a fastening or an adhesion agent. The fastening agent may also work as the adhesion agent. The sheet and the vehicle liquid are stored separately. To use the system, the vehicle liquid is applied either on the target skin area or to the sheet, and the sheet is then applied on the target skin area, so that the vehicle liquid is between the sheet and the skin. When the vehicle liquid is in contact with the sheet and the skin, the local anesthetic impregnated in the sheet dissolves into the vehicle liquid and is delivered into the skin via the vehicle liquid. The system is kept on the skin for sufficient time to significantly reduce the pain. Neuropathic pain includes but is not limited to pain associated with zoster, diabetes-related nerve damage, neuroma (tumor-induced or trauma-induced); nerve damages caused by viral diseases; nerve compression or pinch, and pain or headache associated with occipital neuralgia.

In another embodiment, a method of using some of the aforementioned two-component drug delivery systems for reducing musculoskeletal pain is provided. As described previously, in some of the embodiments, the first component of the system comprises a sheet of a solid and flexible material, and the second component comprises a vehicle liquid. A local anesthetic (such as tetracaine or lidocaine) is impregnated in the sheet, and the vehicle liquid comprises water. The sheet may also comprise a fastening or an adhesion agent. The fastening agent may also work as the adhesion agent. The sheet and the vehicle liquid are stored separately. To use the system, the vehicle liquid is applied either to the sheet or to the target skin area, and the sheet is then applied to the skin area, so that the vehicle liquid is between the sheet and the liquid. When the vehicle liquid is in contact with the sheet and the skin, the local anesthetic impregnated in the sheet dissolves into the vehicle liquid and is delivered into the skin via the vehicle liquid. The system is kept on the skin for a pre-determined period of time before being removed. This process may be repeated once or multiple times a day for days or weeks. Musculoskeletal pain includes but is not limited to pain associated with osteoarthritis; rheumatoid arthritis; myofacial pain; carpal tunnel syndrome; complex regional pain syndrome; tennis elbow; soft tissue and bone injuries such as a sprained ankle, knee, shoulder, wrist, elbow, back; and spondylitis. Musculoskeletal pain also includes pain in bones and joints with any or unknown cause, such as neck, knee, spine, or back pain with any or unknown cause. In treating musculoskeletal pain with the system, significant pain reduction may or may not be achievable with a single application of the system. It is possible that multiple applications over a period of hours, days, or even weeks, are needed before significant pain reduction can be achieved. In those cases, the length of each application may not necessarily be designed to be long enough to produce instantaneous pain reduction, but to deliver sufficient amount of the drug each time (e.g., at least 30 minutes, 30 minutes, 60 minutes; or 2 hours for tetracaine; 2-12 hours for other local anesthetics) so that significant pain reduction is achieved after several applications. In some cases, the system is such that the tetracaine impregnated in said sheet has a sufficient quantity per cm² and a sufficient dissolution speed into the appropriate quantity of said vehicle liquid placed between said sheet and a normal human skin area to be able produce anesthesia in said normal human skin within 120 minutes, or even within 60 minutes, under normal ambient conditions. Although the purpose of these treatments is to reduce musculoskeletal pain instead of to produce skin anesthesia, the time it takes to produce skin anesthesia is a measure of the tetracaine's dermal permeation rates and can be used to gauge the speed of tetracaine delivery or the quantity of tetracaine delivered.

After one application of the tetracaine delivery systems of the current invention, sufficient amount of tetracaine may still exist in the sheet for another application to achieve a desired anesthetic or analgesic effect. Therefore, one of the embodiments of the current invention provides a method of using the sheet and liquid combination system comprising tetracaine for obtaining an anesthetic or analgesic effect, as described in many places in the current application, except the user uses the sheet one or more times.

Although the systems of the current invention that comprise a local anesthetic, such as tetracaine, are capable of producing anesthetic or analgesic effects in intact skin or tissues close to intact skin surface, they can also be used to treat pain in compromised skin, such as scalded skin or mucosal tissues. If the skin area's stratum corneum layer is completely destroyed, such as badly scalded or burned skin, the two-component systems comprising a local anesthetic, as described above, can provide longer-lasting pain relief with a lower risk of local anesthetic overdose than simply applying a local anesthetic solution (e.g. 1% lidocaine hydrochloride solution) onto the wounded skin. That is because the local anesthetic in a typical solution, in which the local anesthetic is completely dissolved, can be quickly absorbed by the capillary blood vessels in the wound that are directly exposed to the solution, while the local anesthetic in the system of the current invention has to dissolve from the sheet and into the vehicle solution or the bodily fluid oozing out of the wound, which takes time. To further extend the drug release time from the sheet and reduce the over exposure risk, the local anesthetic can be incorporated in an ion-exchange resin to form a local anesthetic-ion exchange resin complex which is impregnated in the sheet. When applied on the wounded skin, the local anesthetic molecules can only be exchanged out of the complex by ions in the bodily fluid or the vehicle solution, one ion for each local anesthetic molecule. Because the bodily fluid or the vehicle liquid has limited supply of ions, and replenishing the ions used in exchanging out the local anesthetic molecules takes time, the release rate of the local anesthetic from the sheet is more even over time and the delivery of the local anesthetic into the wounded skin is more sustained over time. Such a system can be beneficially used to treat pain in situations such as burns or deep scalding wounds, or trauma caused by accidents or war acts.

In some of the applications using the embodiments of the current invention, such as aforementioned treatment of musculoskeletal pain and neuropathic pain, the systems of the current invention may be used with localized heat for achieving deeper penetration of the drug into the tissues. For instance, a tetracaine-impregnated sheet, such as one described in some of the Examples below, can be applied over the knee of a patient suffering from pain associated with arthritis, with the vehicle liquid placed between the sheet and the knee skin. A heat generating device, such as a ThermaCare brand air-activated heatwrap, can be placed over the sheet already on the knee. The local heat can increase the skin temperature and likely make the tetracaine penetrate deeper into the knee tissues, which may mean better pain relief.

In another embodiment, a method for reducing pain associated with sores in the oral cavity is provided. The system comprises the first component of a sheet of a solid and flexible material, and the second component is the saliva of the patient. A local anesthetic such as tetracaine or lidocaine is impregnated in the sheet. The sheet may also comprise a barrier film, a fastening agent, and/or an adhesion agent. The fastening agent may also work as the adhesion agent. To use the system, the sheet is applied over the sore area in the oral cavity. The saliva naturally present on the sore surface serves the vehicle liquid. The local anesthetic impregnated in the sheet dissolves into the saliva between the sore surface and the sheet and is delivered into the sore tissue. The pain associated with the sore can be significantly reduced within a few minutes.

In general, the systems of the current invention separate elements of a dermal drug delivery system into two or more components to avoid incompatibility or to gain other benefits, and provide methods for the components to be joined prior to or during the drug delivery application to deliver the drug at sufficient rates to achieve the desired clinical effect. The term “incompatibility” in the current invention means when the elements are incorporated in one formulation or are in contact with each other in another way during storage, at least one of them or the formulation itself becomes chemically or physically unstable to the point that a shelf life of at least one year cannot be achieved at room temperature (based on US FDA standards).

In some embodiments of the current invention, the rationale behind storing the sheet and fluid separately and combining them shortly before or at the application is not to avoid incompatibility, but to gain other benefits. In some such embodiments, a local anesthetic (e.g. lidocaine or tetracaine) is impregnated into a polyurethane film by soaking the film in a local anesthetic solution (see Example 38). When this film is applied to a wound surface, the local anesthetic in the film diffuses into the bodily fluid on the wound surface and then into the wound tissue, reducing the pain associated with the wound. In this system, the bodily fluid of the wound surface is used as the “liquid” part of the sheet and liquid combination system. Additional fluid, such as water, may also be used if more fluid is desirable. The advantages of this system include sustained local anesthetic release into the wound without quick absorption of the drug into systemic circulation, which would happen if a local anesthetic solution is simply applied to the wound surface, due to the open capillary blood vessels in the wound which quickly absorb the drug into systemic circulation. Further advantages include protection of the wound surface from infectious substances and breathability provided by the breathable nature of the polyurethane film. Drugs other than local anesthetics, such as anti-infection agents including, but not limited to, chlorhexidine, can also be impregnated into the film and be used to treat the wound.

In treating musculoskeletal pain in a joint with the sheet and liquid combination system of the current invention, the sheet applied on the joint may need support (in addition to the adhesive agent) to stay adhered to the skin. The joint's movement may have the tendency to cause the sheet and the skin to separate. Therefore, in some of the embodiments of the current invention, the sheet, after being applied to the skin with the vehicle liquid, is wrapped with a wrapper to help keep the sheet in contact with the skin. It is desirable that such a wrapper is made of a breathable material, such as, without limitation, an elastic fabric material (i.e. Ace bandage), whose MVTR is much higher than that of the sheet (such as MVTR higher than 10,000 g/m²/24 hour). In this way, the dry-ending MVTR feature of the sheet may be maintained.

Some embodiments of the current invention are related to a sheet for delivering tetracaine into human skin, comprising at least 0.1 mg tetracaine/cm², wherein said sheet is free of water and said sheet's MVTR is lower than 5,000 g/m²/24 hours, and preferably lower than 2,000 g/m²/24 hours, and can be in the range of between 200 and 10,000 gram/m²/24 hours, 600 to 6,000 gram/m²/24 hours, or 200 to 2,000 gram/m²/24 hours. This sheet can further comprise a lateral diffusion layer which can be a layer of fabric material, such as woven gauze, non-woven absorbent fabric material, paper, open-cell foam, and cloth. The sheet's MVTR property can be provided by a barrier film, such as polyurethane film. The barrier film and the fabric material layer can be laminated together with heat or adhesive. The sheet can also comprise a fastening agent for fastening the active drug and other ingredients onto the sheet. The sheet can further comprise an adhesion agent (such as poly vinyl alcohol) for facilitating the adhering of the sheet, when combined with a vehicle liquid, on to the target skin area. When such a sheet is applied on normal human skin alone or without a vehicle liquid comprising water, it cannot produce anesthesia in said normal human skin within 120 minutes under normal ambient conditions. However, when such a sheet is applied on normal human skin with 25 mg water/cm² between said sheet and said skin, it can produce anesthesia in said normal human skin within 120 minutes under normal ambient conditions. The sheet may further comprise a vasoconstriction agent such as one selected from the group of lidocaine, ephedrine, epinephrine, oxymetazolin, tetrahydrozoline, xylometazoline, phenulphrine, tyramine, naphazoline, caffeine, isoprenaline, pseudoephedrine, orciprenaline, salbutamol, terbutaline.

The sheet and liquid combination systems of the current invention for delivering tetracaine or other local anesthetic into the skin can also include a vasoconstriction agent. A vasoconstriction agent delivered into the skin along with the local anesthetic can reduce the blood flow in the skin area and reduce the speed of clearance of the local anesthetic agent from the skin. As a result, the analgesic or anesthetic effect will last longer. This is an important advantage for pain control applications, because it can potentially reduce the frequency of application which is more convenient to patients and more cost effective. Vasoconstriction agents are molecules capable of constricting the blood vessels, as commonly known in the medical community. They include, without limitation, lidocaine, ephedrine, epinephrine, oxymetazolin, tetrahydrozoline, xylometazoline, phenulphrine, tyramine, naphazoline, caffeine, isoprenaline, pseudoephedrine, orciprenaline, salbutamol, and terbutaline.

In some of the aforementioned applications, only the systems in which the local anesthetic is impregnated in the sheet is used in illustrating how some of the embodiments can be used in the medical applications. However, that is only for example purposes. Other aforementioned systems (e.g. the active drug is in the vehicle liquid) may also be used for the same purposes.

The current invention is also related to a novel manufacturing method of a flexible sheet impregnated with a drug. In typical manufacturing of drug-in-adhesive patches, a thin layer of a mixture of the drug, a pressure sensitive adhesive and volatile solvent is cast onto a backing film. Since the amount of the drug in the patch is proportional to the thin cast layer's thickness, the thickness must be precisely controlled, which can demand high precision of the machinery's design and operation. In the current invention, the preferred manufacturing method involves a different way to dispense the drug onto the sheet. The drug and the fastening agent are dissolved in a volatile solvent. The solution is then dispensed onto the sheet with a volume displacement method, for example with a multi-channel pipette array in which each of the pipettes in the array dispenses a pre-determined volume of the solution by volume displacement in each movement. For example, 300 pipettes can be arranged to form a 10×30 evenly-spaced rectangular array to cover a 10 cm×30 cm area. With each movement of the volume displacement, each pipette dispenses 40 microliters of the solution onto the sheet, so that 12,000 microliters of the solution is dispensed on 300 cm² of the sheet with each movement of the volume displacement. The side of sheet receiving the solution preferably is a material very absorbent to the solution (lateral diffusion layer), such as the gauze side of a gauze-tape laminate sheet. The dispensed solution can thus be quickly absorbed into the absorbent material and flows into even distribution within said sheet of absorbent material. For example, each of the 40 microliter drops of the solution dispensed on the sheet can flow laterally into surrounding areas, so that an even distribution of the solution will be on the sheet some time (e.g., within 30 seconds) after the 300 drops of the solution are dispensed on the sheet. The volatile solvent in the solution is then evaporated off, preferably by passing the sheet through a heating chamber, leaving only the drug and the fastening agent on the sheet. Because it is easier to achieve precise solution dispensing by volume displacement or weighing (in comparison with precisely controlling the thickness of adhesive cast layer in typical drug-in-adhesive patch manufacturing), the sheet can be made at low cost. The phrase “even distribution within said sheet of absorbent material” and the like means the distribution of the drug in the sheet is even enough that no area of the sheet contains no drug, or, if the drug is a local anesthetic, that the skin area treated with the sheet and proper vehicle liquid is relatively evenly anesthetized. “Even distribution” does not necessarily mean the drug quantity per unit area is exactly the same everywhere in the sheet.

### EXAMPLES

In many of the examples below, the ability of the system to numb human skin is used to gauge the effectiveness or stability of tetracaine-impregnated sheets. It should be pointed out that this measurement was only used as a surrogate to measure the rate and/or quantity of delivery of tetracaine into the skin. The ultimate purpose of the sheets of the current invention that comprise tetracaine can be numbing the skin or other than numbing the skin, such as treating musculoskeletal pain, as mentioned previously.

### Example 1

The following system for providing skin anesthesia or analgesia comprising a sheet and a vehicle liquid was made and used as an exemplary embodiment of the current invention.

(1) In this and other examples, tetracaine or TC means tetracaine base, unless specified otherwise. Tetracaine (base), USP (Spectrum Chemical) was dissolved in an isopropyl alcohol:water solution (70:30 by volume, rubbing alcohol, Western Family brand) to obtain a 10% tetracaine (by weight) solution. In this and other Examples, “70% isopropyl alcohol” or “70% isopropyl alcohol solution”, or rubbing alcohol, means Western Family brand rubbing alcohol, which is 70% isopropyl alcohol, 30% water solution by volume. (2) A sheet of gauze (DuSoft® brand Non-Woven Sponge, Dumex, No. 84122, single ply) was placed on the adhesive side of a poly urethane tape (Tegaderm Tape, 3M, purchased from Ortho-Med) to form a laminated sheet. (3) 0.72 gram of the 10% tetracaine solution was evenly dispensed onto 30 cm² of the gauze side of the laminated sheet. The solution was absorbed into the gauze part of the sheet. The sheet was then placed in a heating chamber (temperature cycled in an approximate range of 40-50° C.) for about 30 minutes to let the isopropyl alcohol and water completely evaporate. Each cm² of the dried sheet contained 2.4 mg tetracaine. (4) Separately, a vehicle liquid containing 0.5% Carbopol 981, NF in water, with the pH adjusted to about 7 with NaOH (approximately 0.23% NaOH), was made. The vehicle liquid was a clear, viscous but flowable solution.

To test the system's effect, a thin layer of the vehicle liquid was applied with a thin wood stick to a skin area of approximately 5 cm² on a human subject's left forearm. The thickness of the vehicle liquid layer was such that it was barely thick enough to form a continuous layer. A piece of the tetracaine-containing sheet, approximately 3 cm², was then applied onto the vehicle liquid layer, with the gauze side in contact with the vehicle liquid. The sheet was gently massaged to ensure good contact. The vehicle liquid provided enough adhesion between the sheet and skin (mainly due to the presence of Carbopol 981 as the adhesion agent) so that the sheet stayed on the skin for the entire 60 minute test period. Sixty minutes after the sheet was applied, the sheet was removed from the skin. The skin area that was covered by the vehicle liquid and the sheet was anesthetized (deeply numb). The skin area was also dry when the sheet was removed, suggesting that all the water in the vehicle liquid had evaporated through the sheet.

In the above system, tetracaine was not subject to hydrolytic degradation before it was brought into contact with the vehicle liquid because it existed in the sheet that contained no water. The tetracaine-impregnated sheet can thus be stored at room temperature and has a shelf life of at least two years. When the sheet was applied on the vehicle liquid layer which was on the skin area, the tetracaine in the sheet dissolved into the vehicle liquid which delivered the tetracaine molecules into the skin. The vehicle liquid, after dissolving the tetracaine in the sheet, had a pH high enough to keep a sufficient portion of the tetracaine molecules unionized to facilitate the achievement of anesthesia within 60 minutes. Unionized tetracaine molecules have better skin permeability than ionized tetracaine molecules.

Since the permeation of the tetracaine into the skin stops or slows down greatly when all the water is evaporated, the system in this embodiment has the safety feature of not delivering the drug or delivering the drug at much reduced rates after the desired application period is over.

### Example 2

The following system for providing skin anesthesia or analgesia comprising a sheet and a vehicle liquid was made and used as an exemplary embodiment of the current invention.

Step 1. Ten gram polyvinyl alcohol (PVA, sample from Amresco, molecular weight 30,000 to 50,000) was placed in 90 gram distilled water and heated to approximately 70° C. with periodical stirring until a homogeneous solution was obtained, yielding a 10% PVA (by weight) solution.

Step 2. Five gram of the 10% PVA solution made in Step 1 was added to 6 gram rubbing alcohol. The mixture was shaken until a homogeneous solution was obtained.

Step 3. 0.58 gram tetracaine base, USP (Spectrum Chemical) was added into the solution made in Step 2. The solution was shaken until all tetracaine particles were dissolved, yielding a 5% tetracaine, 4.3% PVA solution (by weight).

Step 4. A gauze sheet (Dusoft Non-woven Sponges, No. 84148, single ply) was horizontally suspended on a lid-less box, so that the sheet was parallel to the ground and the solution-loading area of the gauze sheet was not in touch with any objects. Approximate 4.5 gram (about 5 mL) of the solution made in Step 3 was evenly dispensed onto the 125 cm² the solution-loading area of the gauze sheet with a 5 mL syringe. The sheet was placed in an oven with approximate 50-60° C. temperature for 30 minutes to evaporate off the water and isopropyl alcohol, yielding a gauze sheet impregnated with approximate 1.8 mg tetracaine and 1.5 mg PVA per cm².

Step 5. The gauze sheet made in Step 4 was laminated onto a poly urethane tape (Tegaderm Tape, 3M) using the tape's adhesive. The tetracaine and PVA impregnated sheet was thus completed.

Step 6. A vehicle liquid solution with the following ingredients was made: 5% glycerin, 7% polyvinylpyrrolidone, 0.1% carbopol 981, NF, 0.05% sodium hydroxide, 87.85% distilled water.

The following experiment was performed to test the system made above.

Step 7. A layer of the vehicle liquid made in Step 6 was spread on a human subject's left forearm skin to cover a 3×4 cm area. The thickness of the layer was such that the layer was barely continuous (about 0.2 mm thick).

Step 8. The laminated sheet made in Step 5 (approximate 2×3 cm) was placed on top of the vehicle liquid layer, with the gauze side in touch with the vehicle liquid. A Kleenex tissue was used to tap on the sheet and surrounding area to ensure intimate contact and remove excess vehicle liquid outside the sheet area.

Step 9. Forty five minutes after the commencement of the application, ⅓ of the sheet was lifted and the skin under it was scratched with the end of a straightened paper clip. The skin was anesthetized (deeply numb). The sheet was left on the skin until 120 minutes from the commencement of the application before it was removed. The sheet adhered to the skin very well for the entire 120-minute test period even with movements of the forearm. The skin was still deeply numb when the sheet was removed.

### Example 3

Step 1. Twenty five grams of polyvinyl alcohol (PVA, sample from Amresco, molecular weight 30,000 to 50,000) was placed in 75 grams distilled water and heated to approximately 70° C. with periodical stirring until a homogeneous solution was obtained, yielding a 25% PVA (by weight) solution. This solution is referred to hereafter as “25% PVA solution”.

Step 2. Three and two-tenths grams of the 25% PVA solution made in Step 1 was added to 7.02 grams rubbing alcohol, 0.98 grams distilled water, and 0.8 grams tetracaine base. The mixture was shaken until a homogeneous and clear solution was obtained, yielding a solution with 6.67% tetracaine and 6.67% PVA (by weight).

Step 3. A gauze sheet (Dusoft Non-woven Sponges, No. 84148, single ply) was laminated onto a sheet of the 3M 9832 polyurethane tape using the tape's adhesive (the tape's back release liner was still un-removed).

Step 4. About 5 mL of the solution (about 4.7 grams) made in Step 2 was evenly dispensed onto the gauze side of the sheet (about 160 cm²) made in Step 3 with a 5 mL syringe. The sheet was placed in an oven with approximate 50-60° C. temperature for 30 minutes to evaporate off the water and isopropyl alcohol, yielding a sheet impregnated with approximate 2 mg tetracaine and 2 mg PVA per cm².

Step 5. Separately, a vehicle liquid solution with the following ingredients was made:

0.05% Cabopol 981, N.F., 0.024% sodium hydroxide, 99.926% distilled water.

The following experiments were performed to test the system made above.

Step 6. The vehicle liquid of Step 5 was placed into a spray bottle and sprayed on a human subject's left forearm skin. The skin area was covered with densely populated fine beads of the vehicle liquid.

Step 7. A piece of the laminated sheet made in Step 4 (approximate 2×3 cm) was placed on top of the vehicle liquid already on the skin area, with the gauze side in touch with the vehicle liquid. A Kleenex tissue was used to tap on the sheet and surrounding area to ensure intimate contact and remove excess vehicle liquid outside the sheet area. Forty-five minutes after the commencement of the application, the sheet was lifted and the skin under it was scratched with the end of a straightened paper clip. The skin was deeply numb. The sheet stayed on the skin despite the skin being moved and stretched during the 45 minutes. Although the sheet wrinkled a little bit during the 45 min wear time due to the movement of the arm skin, all the skin area under the sheet was deeply numb, suggesting that slight separation between the sheet and the skin did not affect the anesthesia effect. That may be because at places where the sheet “wrinkles” (i.e., separates from the skin), the vehicle liquid was still on the skin. Enough tetracaine could have dissolved into the vehicle liquid in the first several minutes of the application (before the wrinkles were formed), so that enough tetracaine was delivered into the skin to numb the skin even at places where the sheet wrinkled.

Step 8. In a separate test, a layer of another vehicle liquid (1.6% Carbopol 981, 0.9% sodium hydroxide in water solution) was spread on the forearm skin of a human subject using a thin wood stick. The thickness of the layer was about 0.2 mm (approximately 20 mg/cm²). A piece of the sheet made in Step 4, about 2 cm×3 cm, was placed on top of the vehicle liquid already on the skin area, with the gauze side in touch with the vehicle liquid. A Kleenex tissue was used to tap on the sheet and surrounding area to ensure intimate contact and remove excess vehicle liquid outside the sheet area. Forty-five minutes after the commencement of the application, the sheet was lifted and the skin under it was scratched with the end of a straightened paper clip. The skin was deeply numb. The sheet had stronger adhesion to skin during the test than that in Step 7, suggesting more adhesion agent in the vehicle solution, Carbopol 981, did contribute to stronger adhesion between the sheet and the skin.

In the above systems and experiments, PVA was both a fastening agent and an adhesion agent. Carbopol 981, neutralized with sodium hydroxide, was an adhesion agent. The 3M 9832 polyurethane tape was the MVTR control layer of the solid sheet, and the gauze layer was the later diffusion layer and the fluid retention layer. Tetracaine was the active drug, and the liquids in Step 7 and Step 8 were vehicle liquids.

### Example 4

3.04 grams of the 25% PVA solution made in Step 1 of Example 3, 4.55 grams rubbing alcohol, and 0.51 grams tetracaine base were mixed to yield a clear solution containing 6.3% tetracaine and 9.4% PVA. Approximate 6.7 grams of the solution (about 7 mL) was dispensed evenly on to a 190 cm² gauze-3M 9832 laminate sheet (same as the sheet made in Step 3 of Example 3). The sheet was dried in the oven. The dried sheet contained about 2.2 mg tetracaine and 3.3 mg PVA per cm².

The following experiment was conducted to test the wear property and the anesthesia effect of the system using distilled water as the vehicle liquid.

Distilled water was placed into a spray bottle and sprayed on a human subject's left forearm skin. The skin area was covered with densely populated beads of the distilled water, but the water did not quite form a continuous layer.

A piece of the laminated sheet (approximate 2×3 cm), which contained 2.2 mg tetracaine and 3.3 mg PVA per cm², was placed on top of the distilled water already on the skin area, with the gauze side in touch with the distilled water. A Kleenex tissue was used to tap on the sheet and surrounding area to ensure intimate contact and remove excess vehicle liquid outside the sheet area. Forty-five minutes after the commencement of the application, a corner of the sheet was lifted and the skin under it was scratched with the end of a straightened paper clip. The skin was deeply numb. The sheet was allowed to stay on the skin for a total of two hours before it was removed. At the end of the two hour period, the sheet still adhered to the skin very well, and it had to be peeled off the skin. The peeling force needed was slightly higher than that needed to lift a “post-it” stick note from a skin surface. The skin area was numb for at least four hours following the removal of the sheet.

In the system of this Example, there was no adhesion agent in the vehicle liquid. The only adhesion agent in the system was the PVA impregnated in the sheet, which dissolved into the distilled water after the sheet and the distilled water were brought into contact with each other. The PVA also served as the fastening agent.

In this Example, although the distilled water sprayed on the skin only covered the skin area with water beads and did not quite form a continuous layer, the entire skin area under the sheet was completely numb after the test period, even at places not originally covered by the water beads. That was because the gauze layer (lateral diffusion layer) was very absorbent to the vehicle liquid. After the sheet was applied, the water beads absorbed into the gauze layer quickly spread within the gauze layer to make the entire gauze layer “wet”. The entire skin area was thus covered by the vehicle liquid without any “dry” spots. As the result, the entire skin area covered by the sheet was in contact with tetracaine and water. The skin area was numb without a spot that was not numb.

Therefore, another important feature of the systems of the current invention is that the sheet comprises a layer that is absorbent to the vehicle liquid, or is capable of help spread the vehicle liquid after the sheet is applied over the vehicle liquid between the sheet and the skin.

In the gauze-3M 9832 polyurethane tape laminate sheet of this Example, the gauze has practically no resistance to water vapor transmission (equivalent to extremely high MVTR) while the 3M 9832 poly urethane tape has an MVTR of 800 gram/m²/24 hour (according to 3M). The MVTR of the entire laminate sheet thus is very close to 800 gram/m²/24 hour. In this case, the 3M 9832 polyurethane tape functioned as the MVTR control layer.

### Example 5

The sheet in this Example was the same as that in Example 4.

The following experiment was conducted to test if the same sheet can be used multiple times.

Distilled water was placed into a spray bottle and sprayed on the dorsal side of the left hand of a human subject. The skin area was covered with densely populated beads of the distilled water, but the water did not quite form a continuous layer. A piece of the laminated sheet (approximate 2×3 cm), which contained 2.2 mg tetracaine and 3.3 mg PVA per cm², was placed on top of the distilled water already on the skin area, with the gauze side in touch with the distilled water. A Kleenex tissue was used to tap on the sheet and surrounding area to ensure intimate contact and remove excess vehicle liquid outside the sheet area. Sixty minutes after the commencement of the application, the sheet was removed from the skin and the skin under it was scratched with the end of a straightened paper clip. The skin was deeply numb. The sheet stayed on the skin for the entire 60 min test period. The removed sheet was placed on a piece of paper with the gauze side facing up, so that any residual water in the sheet was allowed to evaporate. After about 30 minutes, distilled water was sprayed onto another part of the dorsal side of the hand skin of the human subject. The sheet used in the first test was applied on the skin with the water beads in between the sheet and the skin. After 60 minutes, the sheet was removed from the skin. The skin surface underneath it was deeply numb, and the sheet stayed adhered on the skin for the entire 60 minutes. The same test was repeated for the 3^{th}, 4^{th}, and 5^{th} time (the 5^{th} time was on forearm skin of the human subject). In the 3^{rd} and 4^{th} tests, the skin under the sheet was deeply numb after the 60 minute application, and the sheet stayed adhered to the skin well for the entire 60 minute test durations. In the 5^{th} test, the sheet stayed adhered well to the skin for the entire 60 minute test period, but the skin under the sheet was not completely numb after the 60 minute application. However, the skin became completely numb about 30 minutes after the sheet was removed. Each of the 5 treated skin areas was numb for at least 5 hours.

The fact that the same sheet was able to produce deep skin anesthesia at least 4 times suggests that it is possible to manufacture a sheet that can be used by the patient multiple times. This would reduce the cost to the patients.

The gauze layer in the sheet was the lateral diffusion layer for achieving even distribution of the vehicle liquid on the skin. Therefore, although the sprayed water beads did not cover the target skin area continuously, water covers the entire target skin area continuously after the sheet was applied because water quickly spread laterally within the sheet so the entire gauze layer, and the skin area, was “wet” without a “dry” spot. As the result, the entire skin area covered by the sheet was in contact with tetracaine and water. The skin area was numb without a spot that was not numb.

### Example 6

In a sheet liquid combination system of the current invention, the sheet itself (without the vehicle liquid) doesn't have to be adhesive to the skin because the vehicle liquid itself or the combination of sheet and liquid (as in Examples 4 and 5) can provide the adhesiveness. However, it can be advantageous to have a sheet that is adhesive to the skin even without the vehicle liquid.

In a sheet liquid combination system in which the sheet is adhesive to the skin without the vehicle liquid, the vehicle liquid is still needed to deliver the drug at the desired rates. However, the sheet can be large enough so that its peripheral area can be used to help adhere the sheet to the skin while its central part can be used, in conjunction with the vehicle liquid, to deliver the drug. To use such a system, the patient or caregiver can applied the vehicle liquid over the target skin area, then apply a sheet that is large enough to cover not only the area covered by the vehicle liquid applied but also some surrounding skin area not covered by the vehicle liquid. Thus, the central area of the sheet is over the target skin area covered with the vehicle liquid, but the peripheral area of the sheet is in direct contact with the skin area not covered by the vehicle liquid. In the area where the sheet is over the vehicle liquid, the drug is delivered at the desired rate. However, in the area where the sheet is in direct contact with the skin without the vehicle liquid, the drug is delivered at much slower rate or not delivered at all for practical purposes, as the vehicle liquid is necessary for the drug to be delivered at the desired rate. Because the sheet is adhesive to the skin without the vehicle liquid, the peripheral area of the sheet can serve as a non-drug-delivery adhesive area to help keep the sheet on the skin.

There are several advantages of this system: (1) the adhesion of the sheet on the skin is not totally dependent on the vehicle liquid, so that the sheet can adhere to the skin better and/or for longer duration. (2) for a target skin area of any shape and size, a piece of sheet can be cut so it has such a shape and size that it can have an area for the delivery of the drug into the target skin area as well as an area for adhering the sheet on the skin.

Some transdermal patches have distinct central areas for drug delivery and peripheral adhesive area to hold the patch on the skin. Those patches are not desirable for target skin areas with irregular shapes or sizes. For example, it is difficult to use a drug delivery patch with a 4″×4″ drug delivery area surrounded by peripheral adhesive area to cover a target skin area of 1″×6″. With the current system, the user can simply cut a 3″×8″ strip out of a large sheet, apply the vehicle liquid over the 1″×6″ target skin area, and apply the 3″×8″ sheet over it. The 1″ wide rectangular ring surrounding the 1″×6″ area can serve as the peripheral adhesive. This is an important versatility for target skin areas with irregular sizes and shapes. For example, the skin areas suffering from post herpetic neuralgia for different patients can have vastly different shapes and sizes.

The following system was manufactured and tested on human skin as an example and one of the possible embodiments of this idea.

All percentages are in weight unless specified otherwise.

Step 1. A solution (Solution A) with the following composition was made: 6.4% polyvinylpyrrolidone, USP (PVP, molecular weight 40,000, Amresco), 3.6% poly ethylene glycol 400 (PEG 400, Spectrum Chemical), 90% rubbing alcohol.

Step 2. A second solution (Solution B) with the following composition was made: 6.4% poly vinyl pyrrolidone, USP (PVP, molecular weight 40,000, Amresco), 3.6% poly ethylene glycol 400 (PEG 400, Spectrum Chemical), 6% tetracaine base, USP (Spectrum Chemical), 84% rubbing alcohol.

Step 3. An 6 cm×10 cm gauze (Dusoft Non-woven Sponges, No. 84148, single ply) was placed on a sheet of release liner. Approximately 1.5 gram of the Solution A made in Step 1 was evenly dispensed onto the gauze. The solution-soaked gauze on the release liner was then placed into an oven to evaporate off the isopropyl alcohol and water.

Step 4. Approximately 1.5 gram of the Solution B made in Step 2 was evenly dispensed onto the dried gauze (still on the release liner) made from Step 3. The gauze was again placed into the oven to evaporate off the isopropyl alcohol and water. The dried gauze now had approximately 3.2 mg PVP, 1.8 mg PEG 400, and 1.5 mg tetracaine per cm².

Step 5. A sheet of the 3M 9832 polyurethane tape (with the non-adhesive side on a release liner) was laminated on top of the dried gauze (which was still on another release liner) made in Step 4, with the adhesive side of the 9832 tape adhering to the dried gauze. The dried gauze now was sandwiched between the release liner and the 9832 tape.

Step 6. Separately, a vehicle liquid with the following ingredients was made: 4% PEG 400, 6% PVP, 0.5% carbopol 981, NF, 0.23% sodium hydroxide, 89.27% distilled water.

The following experiment was performed to test the system made above.

Step 7. A layer of the vehicle liquid made in Step 6 was spread on a human subject's left forearm skin to cover a 2×2 cm area. The thickness of the layer was such that the layer was barely continuous (about 0.2 mm thick).

Step 8. A 4 cm×5 cm piece was cut from the laminated sheet made in Step 5, and the release liners were removed. The sheet piece was then placed on top of the vehicle liquid layer already on the skin, with the gauze side in touch with the vehicle liquid. The central area of the sheet piece was over the vehicle liquid, but the peripheral area was in contact with dry skin area. The sheet piece, especially the peripheral area, was gently massaged to ensure good contact. The sheet piece, including the peripheral area, stayed adhered to skin for the entire 45 minute test period.

Step 9. Forty-five minutes after the commencement of the application, the sheet piece was removed from the skin. The skin area that was covered by both the vehicle liquid and the sheet was deeply numb, but the skin area covered by the sheet only without the vehicle liquid was not numb at all.

Step 10. Separately, a 2 cm×2 cm piece was cut from the laminated sheet made in Step 5, and the release liners were removed. The 2 cm×2 cm sheet piece was applied to a human subject's left forearm skin directly without the vehicle liquid. After 90 minutes, the sheet piece still adhered to the skin well. The sheet piece was then removed from the skin. The skin under the sheet piece was not numb at all. This result demonstrated that the presence of the vehicle liquid was necessary to deliver tetracaine at high enough rates to numb the skin.

### Example 7

In this Example, the system for anesthetizing the skin is similar to that in Example 1, except that the sheet also comprises polyvinyl pyrrolidone (PVP) (e.g. PVP with molecular weight of 40,000, available from Amresco) at 2 mg/cm² as both a fastening and an adhesion agent. Once the sheet is brought into contact with the vehicle liquid, PVP would dissolve into the vehicle liquid. The presence of PVP in the vehicle liquid would increase the adhesion between the skin and the sheet.

### Example 8

The system for reducing the pain associated with post herpetic neuralgia is similar to that in Example 4 or 5. Because the target skin area is also suffering from allodynia (hyper-sensitive skin, where even a light touch can cause excruciating pain), the application of the vehicle liquid on the skin with a Q-tip or stick can cause severe pain to the patient and thus is not desirable. Therefore, an appropriate quantity of the vehicle liquid is applied on the sheet instead of skin. The sheet is then applied on the target skin area, with the side with the vehicle liquid in contact with the skin. More specifically, the vehicle liquid is spread on the sheet with a spatula in a quantity of approximately 20 mg/cm². The sheet is then applied onto the skin area suffering from the pain associated with post herpetic neuralgia. The sheet is maintained on the skin area for 60-240 minutes before it is removed. Significant pain reduction would start within the 60 minute application time, and would last up to 6-10 hours (in non-facial skin) after the sheet is removed from the target skin area. The skin area would be dry when the sheet is removed, since water in the vehicle liquid under the sheet would have evaporated through the sheet and the sheet would have the “dry-ending” MVTR. To obtain around-the-clock pain control (or close to it), the patient would use the system every 8-12 hours. If the application time is 60 minutes, the skin area is covered with the sheet for only two to three hours in a 24 hour period. Around-the-clock pain control with only two to three hours a day of skin occlusion is an important advantage, because it means little, if any, skin irritation, discomfort, or inconvenience would occur.

### Example 9

In this Example, the patient uses the same system to treat post herpetic neuralgia as described in Example 8, except the patient re-uses the same sheet at least twice, in a manner similar to that described in Example 5.

### Example 10

This Example describes a three-component system for reducing neuropathic pain with capsaicin without the skin burning sensation.

Qutenza® capsaicin patch is approved for treating pain associated with post herpetic neuralgia. It may also be effective in treating other neuropathic pain, such as diabetes-induced neuropathic pain, in the skin or tissues close to the skin surface. However, the high concentration of capsaicin in the patch itself can cause a severe burning sensation and pain. This is why the patient's skin has to be pre-treated with a local anesthetic product, such as an EMLA cream, before the Qutenza® capsaicin patch is applied. The patient typically has to wait in the clinic for one hour or longer for the pre-treatment, which typically needs to be performed by clinic personnel, to produce the numbing effect. After the treatment when the capsaicin patch is removed, the pain control by the local anesthetics (lidocaine and prilocaine in the case of EMLA) often does not last long enough (typically no longer than 1-2 hours), so that patients can suffer from post-treatment burning sensation.

To address this problem with an embodiment of the current invention, a three component system is made. The first component is an oil-in-water emulsion cream containing tetracaine hydrochloride (in the aqueous phase, with a typical concentration of 3% of the total formulation weight), soybean oil (the oil phase, with a typical concentration of 30% of the total formulation weight) and polyvinyl alcohol (PVA) (in the aqueous phase, with a typical molecular weight 20,000-60,000, and a typical concentration of 10% of total formulation weight). Soybean oil is a good solvent for capsaicin. “Good solvent for capsaicin” in this disclosure means a solvent with a capsaicin solubility of at least 100 mg/Liter. Vegetable oils, such as soybean oil, are good solvents for capsaicin. The pH of the cream (the aqueous phase) would be 5 (to be achieved with an acid, such as hydrochloric acid, if necessary, but is not pH buffered), so that the tetracaine in the formulation is stable enough to give the formulation a shelf life of at least 12 months at room temperature. The oil phase is emulsified in the aqueous phase with the help of an emulsifying agent. The viscosity of the cream would be such that a 1 mm thick layer of the cream can be easily applied and maintained on the target skin area. The second component is a fabric sheet material impregnated with sodium borate and a pH modifying agent (base). The fabric sheet is designed to have a pre-determined capsaicin permeability. The 3^{rd} component is a capsaicin patch, similar to the Qutenza® capsaicin patch. To use the system, a layer of the cream is applied on the target skin. The second component sheet is then applied on top of the cream layer. The 3^{rd} component capsaicin patch is then applied on top of the 2^{nd} component sheet. Once this configuration is in place, the pH modifying agent in the sheet (the 2^{nd} component) would dissolve into the cream layer and increase its pH to 7.5 or higher, thus making the tetracaine in the cream layer much more skin-permeable (compared to when the pH is low). Tetracaine would permeate into the skin and would numb it within 60 minutes. Meanwhile, the sodium borate in the 2^{nd} component sheet would also dissolve into the cream layer and crosslink the PVA, so that the cream layer is converted into to soft solid layer within 60 min. The capsaicin in the patch would have to permeate through the 2^{nd} component fabric sheet and across the thickness of the cream layer (mainly utilizing the soybean oil phase in the cream, because capsaicin is very soluble in soybean oil but poorly soluble in water) before reaching the skin surface, but it would eventually reach the skin surface. If the components are designed correctly (e.g. proper fabric sheet capsaicin permeability, thickness of the cream layer, and soybean oil content in the cream), the skin can be numbed by the tetracaine before the capsaicin can cause burning sensation. After the treatment time (e.g. 90-120 minutes), all three components would be removed from the skin. Because the cream layer would already be solidified and attached to the sheet, it is automatically removed when the sheet (and the capsaicin patch) is removed, leaving no messy residue on the skin surface. Because tetracaine can produce a much longer analgesic effect after the dermal delivery is stopped than lidocaine or prilocaine, the post-treatment numbing effect can last as long as 6-12 hours (in non-facial skin) The patient therefore has a much lower chance of suffering from the post-treatment burning sensation. This system would allow the patient to come to the clinic, let the physician or nurse to put the system on the target skin area as described above, and go home. The patient can remove the system himself/herself after a pre-determined time. This approach would avoid the wait in the clinic.

### Example 11

Topical NSAID products such as the ones containing about 1.6% diclofenac as the active ingredient are used to treat pain associated with osteoarthritis of the knee (OA of the knee) and soft tissue injuries such as sprained ankle. For example, Pennsaid diclofenac solution is used to treat pain associated with OA of the knee. However, the patient has to apply 40 droplets of the solution on the knee, 4 times a day. The applied solution can be easily wiped off by objects (e.g. pants) unintentionally. The 4-times-daily application is inconvenient. A system of the current invention designed to mitigate the problem has two components. The first component is a diclofenac solution, similar to or the same as the Pennsaid solution. The second component is a sheet impregnated with an adhesion agent and having a dry-ending MVTR for the diclofenac solution. The sheet is also stretchable (elastic). To use the system, the user applies about 2 mL of said diclofenac solution on the target knee skin area, and then covers the skin area with the sheet. The adhesion agent (e.g. poly vinyl pyrrolidone or PVA) would dissolve into the diclofenac solution and make it act as an adhesive to properly adhere the sheet onto the skin. To achieve the desired effect, the sheet should be kept adhered to the normal human skin surface under normal ambient conditions for at least 15 min, and preferably at least 60 min. Volatile solvents in the diclofenac solution would slowly evaporate through the sheet. After the desired application time, the sheet would be removed from the skin. In this approach, the diclofenac solution is protected from unintentional removal and premature evaporation of the solvent (which stops the drug delivery), so that more diclofenac is delivered in each treatment. The frequency of treatment in each day can thus be reduced, which is more convenient to the patient and can improve patient compliance.

In this Example, the reason to use the sheet and liquid combination system of the current invention is not to improve the drug's stability. The advantage of using the sheet and liquid combination system in this case is being able to conveniently keep the drug solution on the skin for longer time, so the user doesn't have to wait for the solution to dry on the skin or worry about the solution being removed by clothing.

### Example 12

The system for preventing the pain associated with laser tattoo removal is similar to that in Example 4. To use the system, a vehicle liquid (such as distilled water) is sprayed on the target skin area in a manner similar to that described in Example 4. The sheet is then applied onto the vehicle liquid layer already on the target skin area. The sheet is maintained on the skin area for 90 minutes before it is removed. The skin area would be anesthetized, and the laser tattoo removal procedure can be performed with minimal or no pain to the patient. The skin area would be dry or almost dry when the sheet is removed, since water in the vehicle liquid layer would have evaporated through the sheet and the sheet has the “dry-ending” MVTR

### Example 13

The system for reducing the pain associated with herpes zoster in the acute eruptive phase is similar to that in Example 3. To use the system, the vehicle liquid is spread on the sheet at a quantity of approximately 20 mg/cm². The sheet is then applied onto the skin area suffering from the pain associated with herpes zoster in its eruptive acute phase (with blisters and/or rash). The sheet is maintained on the skin area for 60 minutes before it is removed. Significant pain reduction would start within the 60 minute application time, and would last several more hours after the sheet is removed from the target skin area. The skin area would be dry or almost dry when the sheet is removed, since most or all of the water in the vehicle liquid layer would have evaporated through the sheet and the sheet has the “dry-ending” MVTR.

### Example 14

The system is similar to that in Example 13, except in this case the vehicle liquid is in a fine mist spray bottle and is sprayed either on the target skin area or on the sheet.

### Example 15

The system for reducing the pain associated with carpal tunnel syndrome is similar to that in Example 5. To use the system, the vehicle liquid, which is contained in a spray bottle, is sprayed on the skin of the carpal tunnel area, at a quantity of approximately 20 mg/cm². The sheet is then applied onto the skin area and maintained there for 60 minutes before it is removed. For most patients in most situations, the skin area would be dry when the sheet is removed, since water in the vehicle liquid layer would have evaporated through the sheet and the sheet has the “dry-ending” MVTR. The application can be repeated at a frequency and for a number of times as adequate for significantly reducing the pain of the individual patient. The same sheet may be used multiple times in a manner similar to that described in Example 5.

### Example 16

The system for reducing the pain or headache associated with occipital neuralgia is similar to that in Examples 3-5. To use the system, the vehicle liquid is spread on the target skin area (typically the target skin area is the skin area over or adjacent to the occipital nerve), at a quantity of approximately 20 mg/cm². The sheet is then applied onto the skin area and maintained there for 90 minutes before it is removed. For most patients in most situations, the skin area would be dry when the sheet is removed, since water in the vehicle liquid layer would have evaporated through the sheet and the sheet has the “dry-ending”. The application can be repeated at a frequency and for a number of times as adequate for reducing the pain or headache of the individual patient.

### Example 17

The system for reducing back pain is similar to that in Examples 3-5, except that the vehicle liquid is soaked in a sheet of absorbent fabric, at a quantity of approximately 20 mg/cm², prior to the application. To use the system, the fabric sheet soaked with the vehicle liquid (wet sheet) is placed on the skin under which the back pain exists. The tetracaine-impregnated sheet is then applied over the wet sheet and maintained there for 90 minutes before both sheets are removed from the skin. Tetracaine in the fabric sheet would get dissolved into the vehicle liquid and permeate into the skin. The application can be repeated at a frequency and for a number of times as adequate for reducing the back pain of the individual patient.

### Example 18

The system for reducing pain associated with osteoarthritis of the knee (OA of the knee) is similar to that in Examples 3-5, except that four pieces of the sheet (four sheets) are used for each knee. To use the system, the vehicle liquid is applied on the front, back, and sides of the knee suffering from OA of the knee, at a quantity of approximately 20 mg/cm². One sheet each is applied to each of the front, back, and two sides of the knee, over the applied vehicle liquid, so that four pieces of the sheet are used for each knee. The sheets are maintained there for 90 minutes before removal. The application can be repeated at a frequency and for a number of times as adequate for reducing the pain of the individual patient.

### Example 19

The system and method in this Example are similar to that in Example 18, except in this case the purpose is to treat pain associated with rheumatoid arthritis of the knee.

### Example 20

The system for reducing pain associated with sprained joints, including sprained ankle, knee, or shoulder, is similar to that in Examples 3-5, except the number of pieces of the sheet used for the joint can be varied depending on the size and the curvature of the joint. To use the system, the vehicle liquid is applied on the skin area over the injured joint, at a quantity of approximately 20 mg/cm². One or more sheets are applied to the skin area. The size and number of the pieces of the skeet used are determined by factors such as the size of the joint and the curvature of the skin area, to maximize comfort and minimize the interference with joint movement and potential of separation of the sheet(s) from the skin. The sheet(s) is maintained there for 90 minutes before removal. The application can be repeated at a frequency and for a number of times as adequate for reducing the pain of the individual patient.

### Example 21

In this Example, the systems and methods for reducing back pain, pain associated with OA of the knee, rheumatoid arthritis of the knee, and sprained joints, including sprained ankle, knee, or shoulder are the same as that in Examples 17-20. In addition, a ThermaCare brand air-activated heat wrap is applied over the sheet which is already applied on the skin. The local heating can increase the permeability of the skin to tetracaine and help drive the tetracaine into deeper tissues, which may mean better clinical results.

### Example 22

The systems and medical conditions to be treated with the systems are similar to those in Examples 12-20, except that the drug is lidocaine and the application time can be 2-12 hours, and also can be 5-12 hours.

### Example 23

The system for reducing pain associated with scalded skin is similar to that in Example 4 or 5. To use the system, the vehicle liquid is sprayed on the skin area over the scalded skin area or on the sheet. The sheet(s) is applied on the scalded skin with the vehicle liquid between the sheet and the skin, and maintained there for 60 minutes before removal. The application can be repeated when or if the pain comes back.

### Example 24

The system for reducing pain associated with scalded or burned skin is similar to that in Example 4 or 5 (where the adhesion agent is impregnated in the sheet), except that the local anesthetic agent is lidocaine. In this case, the scalded or burned skin area is so damaged that the stratum corneum layer (the main barrier layer of the skin) is all or mostly damaged, and there is visible bodily fluid oozing out of the area. To use the system, the sheet is directly applied to the skin area without the vehicle liquid, and maintained there for a period of time that offers a good balance between the need of pain control and wound care (i.e. between the need of treating the wound with anti-infection and/or wound healing medication and the need of the wound to be exposed to air). The bodily fluid oozing out of the wound skin would serve as the vehicle solution here to dissolve the tetracaine in the sheet and deliver it into the wounded skin. The adhesive agent impregnated in the sheet would dissolve into the bodily fluid and provide the adhesion for keeping the sheet on the skin.

### Example 25

The system for reducing pain associated with scalded or burned skin is similar to that in Example 24, except that the sheet comprises a lidocaine-ion exchange resin complex instead of un-complexed lidocaine. The scalded or burned skin area is so damaged that there is visible bodily fluid oozing out of the area. To use the system, the sheet is directly applied to the skin area without the vehicle liquid, and maintained there for a period of time that offers good balance between the need of pain control and wound care (i.e. between the need of treating the wound with anti-infection and/or wound healing medication and the need of the wound to be exposed to air). The ions in the bodily fluid exchange out the lidocaine molecules in the lidocaine-ion exchange resin complex impregnated in the sheet, so the lidocaine release is controlled and extended. The patient thus enjoys a longer pain relief effect ad lower risk of lidocaine exposure than he/she would if a lidocaine hydrochloride solution is directly applied to the scalded or burned skin. Optionally, an adhesive agent can be impregnated in the sheet, and can dissolve into the bodily fluid and provide the adhesion for keeping the sheet on the skin.

### Example 26

In this Example, a roll of absorbent paper (or other fabric) is soaked in the vehicle liquid which comprises an adhesion agent and is contained in a container. The roll of absorbent paper is made of many pre-cut pieces that are rolled in a way that when one piece is pulled out from the container, it brings up the next piece. This arrangement is similar to some baby wipe products or Kleenex tissues in a box. Alternatively, the roll of the absorbent paper is a continuous roll with periodical perforation or partial cuts for easy tearing of the pieces along the perforation lines. The container may have an attached sharp edge to facilitating the tearing. To use the system, the user pulls a piece of the vehicle liquid-soaked absorbent paper out of the box and lays it on the target skin area. The user would then apply the drug-impregnated sheet (similar to that in previous Examples) on top of the vehicle liquid-soaked absorbent paper and maintain it there for the desired duration of dermal drug delivery. Once the sheet is on top of the vehicle liquid-soaked absorbent paper which is on the target skin area, the drug impregnated in the sheet would dissolve into the vehicle liquid which helps deliver the drug into the skin. The sheet can have the “dry-ending” MVTR so that at the end of the application period, there is little or no liquid under the sheet.

### Example 27

### Production and Testing of a Laminated Sheet Impregnated with Tetracaine Base (TC) and Polyvinyl Alcohol (PVA)

Step 1. 176.2 gram rubbing alcohol was mixed with 22.4 gram of a 25% PVA solution (25% PVA, 75% water by weight) to form a “blank loading solution”. The blank loading solution contained about 2.82% PVA and had a density of about 0.91.

Step 2. 0.35 g tetracaine base (TC) was added into 49.72 gram of above blank loading solution, and completely dissolved, to form TC Loading Solution One which contained 0.7% TC, 2.8% PVA, and had a density of about 0.91.

Step 3. 15 mL (about 13.65 g) TC Loading Solution One was dispensed onto 190 cm² fabric gauze (single ply, Dusoft 84148 from Derma Sciences) placed on a plastic release liner (3M9956). The solution soaked fabric was allowed to dry overnight at room temperature. This “loaded” fabric had 0.5 mg TC and 2 mg PVA/cm².

Step 4. 22.49 g TC Loading Solution One was mixed with 15.02 g blank loading solution to form TC Loading Solution Two which contained 0.42% TC and 2.8% PVA. Its density was about 0.91.

Step 5. 15 mL (about 13.65 g) TC Loading Solution Two was dispensed onto 190 cm² fabric gauze (single ply, Dusoft 84148 from Derma Sciences) placed on a plastic release liner (3M9956). The solution soaked fabric was allowed to dry overnight at room temperature. This “loaded” fabric had 0.3 mg TC and 2 mg PVA/cm².

Step 6. The air-dried fabric sheets from Steps 3 and 5 were placed into an oven with temperatures cycling between approximately 50 and approximately 60° C. and removed from the oven after about 30 min. to evaporate any remaining solvent not evaporated during the room temperature drying process.

Step 7. The heat dried fabric sheets from Step 6 were laminated to a polyurethane tape (3M9834) using the tape's adhesive. The laminated sheets had 0.5 mg TC+2 mg PVA/cm² and 0.3 mg TC+2 mg PVA/cm², respectively. The sheets made in Step 7 can be used as the sheet in the “Sheet Liquid Combination” of the current invention. The TC was the active drug, PVA was the fastening agent as well as the adhesion agent. The fabric layer had the lateral diffusion function and can serve as the “lateral diffusion layer”. The fabric layer was also the “liquid retention layer. The poly urethane tape was the MVTR control layer.

Step 8. On the day after Step 7, the following skin test was conducted: Distilled water droplets were sprayed onto the dorsal side of a human subject's hand. A piece (1 cm×2 cm) of the laminated sheet made in Step 7 containing 0.3 mg TC+2 mg PVA per square centimeter was placed on the wet skin with the fabric side of the sheet in contact with the skin. In this and other Examples, when a fabric-film laminated sheet is said to be placed on the skin, it always means the fabric side is placed in contact with the skin. A paper tissue was used to tap on the outer face of the sheet and surrounding area of skin to ensure good contact and remove excess water. At 45 min from the start of the application, the sheet was lifted and the skin under it was poked with the end of a straightened paper clip. In this and other Examples, skin anesthesia, or numbness, was tested by the poking or scratching with an end of a straightened paper clip, unless specified otherwise. Similar testing methods are known to those of skill in the art. The skin area was almost completely numb. At 60 min, the sheet was removed from the skin. The skin area treated by the sheet was completely numb. The sheet adhered to the skin very well for the entire 60 min test period.

Step 9. One week after Step 7, the sheets made in Step 7 were each cut into halves and the resulting four pieces of sheets were individually wrapped in aluminum foil. One of the 0.3 mg TC+2 mg PVA/cm² pieces and one of the 0.5 mg TC+2 mg PVA/cm² pieces were placed into a Styrofoam box, which was placed into an oven with temperatures cycling between about 63 and about 68° C. (referred to as 65° C. hereafter for simplicity). The remaining pieces were stored at room temperature. After 11 days, the pieces in the oven were removed from the oven and stored at room temperature. Two days later, all four pieces were tested for their skin anesthetizing ability using a method similar to that in Step 8. The results were as follows:

| | | Skin numbness after |
|---|---|---|
| Sheet | Storage condition | 60 min treatment |
| 0.5 mg TC + 2 mg PVA/ | 11 days at 65° C., 9 days | Not numb |
| cm² | at room temperatures | |
| 0.5 mg TC + 2 mg PVA/ | 20 days at room | Numb* |
| cm² | temperatures | |
| 0.3 mg TC + 2 mg PVA/ | 11 days at 65° C., 9 days | Not numb |
| cm² | at room temperatures | |
| 0.3 mg TC + 2 mg PVA/ | 20 days at room | Numb* |
| cm² | temperatures | |

*The skin area treated by the unheated sheet containing 0.5 mg + 2 mg PVA/cm² felt more profound numbness than that by the unheated sheet containing 0.3 mg + 2 mg PVA/cm², suggesting that the former sheet delivered more TC into the skin.

These results suggest that the lamination adhesive of the 3M9834 tape can interact with the TC formulation after long term storage. By a rule of thumb that drug degradation rate increases by a factor of 3 for every 10° C. storage temperature increase, storage at 65° C. for 11 days is approximately equivalent to storage at room temperature for about 2.5 years. Therefore, the above results suggest that if 3M9834 tape is used in the sheet, the sheet's anesthetizing ability can be compromised after long term storage at room temperature.

### Example 28

As suggested by the results of the previous Example, it is possible that certain adhesives used to laminate the fabric layer to the polyurethane film (lamination adhesives) may interact with tetracaine chemically or physically such that the tetracaine impregnated sheets' ability to produce skin anesthesia is compromised after long-term storage. The following experiments were conducted to select a tape and its adhesive that do not compromise the anaesthetic effect of the tetracaine.

A blank loading solution with the following components was made:

| | |
|---|---|
| 70% isopropyl alcohol solution | 176.2 gram |
| (70% isopropyl, 30% water, by volume) | |
| 25% PVA solution | 22.4 gram |
| (25% PVA, 75% water) | |

The density of this loading solution was about 0.91 and it contained about 2.82% PVA.

Three tetracaine loading solutions with the following compositions were made:

Tetracaine Loading Solution A: 1.4% tetracaine base, 98.6% blank loading solution.

Tetracaine Loading Solution B: 0.7% tetracaine base, 99.3% blank loading solution.

Tetracaine Loading Solution C: 0.42% tetracaine base, 99.58% blank loading solution.

Fifteen mL Tetracaine Loading Solution A was dispensed evenly onto 190 cm² of an absorbent fabric (Derma Sciences Dusoft 84148, single ply) resting on a release liner. The solution soaked fabric was placed on a flat surface in an oven with temperatures cycling approximately between 62 and 68° C. for 45 min to evaporate off the solvents. The dried fabric contained 1 mg tetracaine and 2 mg PVA per square centimeter.

The same procedure was repeated with Tetracaine Loading Solutions B and C, respectively, to produce fabric sheets impregnated with 0.5 mg tetracaine and 2 mg PVA per square centimeter, and 0.3 mg tetracaine and 2 mg PVA per square centimeter, respectively.

Each of the dried fabrics above was cut into three equal parts which were laminated to three polyurethane tapes: 3M9832, 3M9834, 3M9948, respectively, with the dried fabric adhered to the adhesive side of the tape. Nine different laminated sheets were thus produced.

Each of the nine laminated sheets was cut into half. One of the halves was wrapped in aluminum foil and stored at room temperature. The other halves were wrapped in aluminum foil and placed into an oven at a temperature cycling between approximately 62 and approximately 68° C. (for simplicity, the 62-68° C. cycling temperatures will be referred to hereafter as 65° C.).

After 11 days, all the samples were removed from the oven and some of the 18 samples (9 stored at room temperature and 9 stored at 65° C.) were tested on a human subject's skin for skin anesthesia with the following method: A 1 cm×2 cm piece of each of the test sheets was applied onto the skin surface of the subject which was covered with fine distilled water droplets sprayed from a spray bottle. A paper tissue was used to gently tap the sheet and surrounding area to ensure good sheet-skin contact and remove water from outside the sheet area. After 60 minutes, the sheet was removed and skin anesthesia was tested by poking with the end of a straightened paper clip. The samples tested, the skin sites, and anesthesia results are summarized in the following table.

| | | | | | Skin |
|---|---|---|---|---|---|
| Tetracaine/ | | Type of | Storage | | numbness |
| cm² | PVA/cm² | tape in | temp- | Skin | after 60 min |
| (mg/cm²) | (mg/cm²) | laminate | erature | site | application |
| 0.3 | 2 | 3M9832 | 11 days at | Dorsal | Numb |
| | | | 65° C. | hand | |
| 0.3 | 2 | 3M9834 | 11 days at | Dorsal | Not numb |
| | | | 65° C. | hand | |
| 0.3 | 2 | 3M9832 | Room | Dorsal | Numb |
| | | | | hand | |
| 0.5 | 2 | 3M9832 | 11 days at | forearm | Numb |
| | | | 65° C. | | |
| 0.5 | 2 | 3M9834 | 11 days at | forearm | Not numb |
| | | | 65° C. | | |
| 0.3 | 2 | 3M9948 | 11 days at | Dorsal | Not numb |
| | | | 65° C. | hand | |
| 1 | 2 | 3M9834 | 11 days at | Dorsal | Not numb* |
| | | | 65° C. | hand | |
| 0.3 | 2 | 3M9948 | Room | Dorsal | Not |
| | | | | hand | numb** |
| 0.3 | 2 | 3M9832 | Room | Dorsal | numb |
| | | | | hand | |

*This skin site was numb when tested at 120 minutes (60 minutes after the sheet was removed from the skin site), suggesting that the anesthetizing ability had not been totally destroyed by the storage at 65° C. for 11 days.
**The skin site was numb when tested at 180 min.

One can see from the above results that: (1) sheets with the 3M9832 tape, regardless of the storage temperature or tetracaine quantity, maintained anesthetizing ability; (2) sheets with 3M9834 tape, after 11 days at 65° C., regardless of tetracaine quantity, lost anesthetizing ability; (3) sheets with 3M9948 tape lost the anesthetizing ability after 11 days storage at 65° C., and similar sheets lost much anesthetizing ability after about 20 days of room temperature storage.

The purpose of storing the samples at 65° C. was to estimate their long term (years) stability using a much shorter time period. As a rule of thumb in drug stability tests, the rate of drug loss increases by a factor of three for every 10° C. that the storage temperature is increased. Therefore, the rate of drug loss at 65° C. is approximately 80 times higher than that at 25° C., and storage at 65° C. for 11 days is equivalent to approximately 2.4 years of storage at 25° C. Although this is an estimation, it was effective to demonstrate that different lamination adhesives can have different impacts on the laminated sheets' long term stability. These results suggest that the sheets with 3M9834 or 3M9948 tapes will likely lose anesthetizing ability after 2.4 years of storage at room temperature, while sheets with 3M9832 tape will not.

According to 3M, the 3M9832 and 3M9834 tapes use the same polyurethane film, but different adhesives. It follows that the loss of anesthetizing ability in sheets with 3M9834 must be caused by 3M9834's adhesive. It is likely that the loss of anesthetizing ability in sheets with 3M9948 was also caused by its adhesive. Since all three tapes are medical grade tapes and have otherwise similar properties, it is surprising that only 3M9832's adhesive does not cause the loss of anesthetizing ability.

### Example 29

### Heat Lamination of Rayon-Polyester Blend Fabric to Polyurethane Film

Example 28 demonstrated that the lamination adhesive can potentially interact with the tetracaine formulation to compromise anesthetizing ability. While selecting a lamination adhesive that does not compromise the anesthetizing ability is one approach to dealing with this potential problem, another is to laminate the fabric layer and the barrier film together by heat, avoiding the use of lamination adhesive completely. The following Example demonstrates such a heat lamination process.

Heat press used: Seiki Technology, Type SK-HP3

Materials to be heat laminated: a rayon polyester blend fabric (Derma Sciences Dusoft 84122, same material as Dusoft 84148, single ply) and a polyurethane film (3M 9832F).

For each of the tests (with different heat press heating temperature settings) the fabric and film were assembled as described in Example 33 and heat pressed. The heating temperature and duration setting, along with the observed results, are summarized in Table 1.

TABLE 1

| Heating | Heating | |
|---|---|---|
| temp. setting | Duration setting | Observation |
| 260° F. | 2 seconds | Fabric and film had a weak bond, |
| | | but were still separable |
| 270° F. | 2 seconds | Fabric and film had a weak bond, |
| | | but were still separable |
| 280° F. | 2 seconds | Fabric and film had a weak bond, |
| | | but were still separable |
| 290° F. | 2 seconds | Fabric and film bonded, but still |
| | | separable |
| 300° F. | 2 seconds | Fabric and film bonded, barely |
| | | separable |
| 310° F. | 2 seconds | Fabric and film bonded, difficult to |
| | | separate, but still separable |
| 320° F. | 2 seconds | Fabric and film bonded, very |
| | | difficult to separate |
| 330° F. | 2 seconds | Fabric and film bonded, not |
| | | separable |

No film damage was observed in any of the above tests.

Separately, tests were conducted for heat laminating the same rayon polyester blend fabric (Derma Sciences Dusoft 84148) with a different polyurethane film (America Polyfilm, Inc. MT1001-AM). The method was similar to the previous tests. The results are summarized in Table 2.

TABLE 2

| Heating | Heating | |
|---|---|---|
| temp. setting | Duration setting | Observation |
| 330° F. | 2 seconds | Bonded, but slight damage |
| | | to the film occurred |
| 300° F. | 2 seconds | Bonded, almost no damage |
| | | to the film observed |

These results suggest that the rayon polyester blend fabric can be laminated to a polyurethane film with heat, but proper heating temperature and duration must be used. The lamination of the fabric and the film with heat eliminates the need to use adhesive for lamination, which could mean the avoidance of potential adverse interactions between the drug formulation and the lamination adhesive, as well as lower material costs since a film coated with adhesive is typically significantly more expensive than the film purchased alone.

### Example 30

### Heat Lamination of Polyurethane Film to a Fabric Already Loaded with Tetracaine and PVA

A piece of a rayon polyester blend fabric (Derma Sciences Dusoft 84148) was loaded with 0.3 mg TC and 2 mg PVA per square centimeter. The method of loading the TC and PVA was similar to that described previously.

The loaded fabric piece was laminated to a polyurethane film (3M 9832F) with the heat press (Seiki Technologies, Type SK-HP3) with heating temperature setting of 330° F. and heating duration of 30 seconds. The heat lamination process was similar to that described previously.

The following skin test was conducted to determine if the 330° F. heating for 30 seconds destroyed the anesthetizing ability of the sheet: fine water droplets were sprayed onto the forearm skin of a human subject. A 1 cm×2 cm piece of the above-described heat-laminated sheet was applied on the wet skin. After 60 min, the sheet was removed from the skin. The skin area was dry and deeply numb (as determined by poking with an end of a straightened paper clip).

The above experiment was repeated with the same fabric loaded with 0.5 mg and 2 mg PVA per square centimeter. After 60 min treatment with the laminated sheet, the skin area was also dry and deeply numb.

These results suggest that heating the TC+PVA formulation already coated on the fabric at 330° F. for 30 seconds does not destroy the anesthetizing ability. It should be noted that this does not necessarily mean that no TC was degraded during the heating process. It is possible that some TC was destroyed in the heating process. Despite this, however, enough TC survived to maintain the desired anesthetic effect.

### Example 31

### Effect of Different Degrees of Occlusion on Skin Anesthesia Duration

A previously made fabric (Derma Sciences Dosoft 84148 rayon polyester blend) loaded with 1.98 mg TC and 4.74 mg PVA per square centimeter was laminated to a polyurethane tape (3M 9832) using the tape's adhesive. Fine water droplets were sprayed onto the forearm skin of a human subject. Two 1 cm×2 cm pieces of the laminated sheet were applied on the wet skin. One of the two pieces was then covered with a piece of Scotch® tape (3M) to produce better occlusion (lower MVTR). After 60 min, both sheets were removed from the skin. The skin area under the sheet that was covered by the Scotch® tape was more moist than the other skin area, likely as a result of better occlusion. Both skin areas were deeply numb (as tested by poking with a straightened paper clip). The numbness of the skin areas was tested at the following time points:

6 hours (5 hours after sheet removal): both areas deeply numb.

7 hours (6 hours after sheet removal): both areas deeply numb.

8 hours (7 hours after sheet removal): both areas lost most of the numbness. There is no difference in numbness between the two areas.

These results suggest that the occlusion produced by the 3M 9832 tape is good enough in terms of producing the anesthesia effect for an extended period of time, and better occlusion (lower MVTR) does not produce longer lasting anesthesia.

### Example 32

### Effect of High Temperature Storage on Anesthetizing Ability of the Sheet

The following sheets were made before the skin test:

Sheet 1: A Dusoft 84148 fabric was loaded with 0.3 mg tetracaine base (TC) and 2 mg PVA/cm². The process of loading the TC and PVA was the same as that described previously. The dried loaded fabric was laminated to the 3M9832 tape using the tape's adhesive. The laminated sheet was stored in an oven with temperatures about 155° F. for at least 30 days.

Sheet 2: Same as Sheet 1, except that the fabric with loaded with 0.5 mg TC and 2 mg PVA per cm². The sheet was also stored in the oven with temperatures about 155° F. for at least 30 days.

Sheet 3: A Dusoft 84148 fabric loaded was with 0.5 mg tetracaine base (TC) and 2 mg PVA/cm². The dried loaded fabric was not laminated to any film and was stored in the oven with temperatures about 155° F. for at least 30 days. The loaded fabric was then laminated to the 3M9832 tape using the tape's adhesive just before the skin test below.

The following skin test was conducted:

Water was sprayed on the skin of the back of a human subject's hand. Each of the three sheets above, about 1 cm×2 cm, was placed on the wet skin, with the fabric side in contact with the skin. After 60 min (t=60 min), the sheets were removed from the skin. Skin anesthesia was measured by poking the skin area with the end of a straightened paper clip at each test time point, with the following results:

| Time (from start of the | | | |
|---|---|---|---|
| sheet application) | Sheet 1 | Sheet 2 | Sheet 3 |
| 60 min | Not numb | Numb | Numb |
| 150 min* | Numb | Numb | Numb |
| 210 min | Not numb | Numb | Numb |
| 240 min | Not numb | Numb | Numb |
| 270 min | Not numb | Not numb | Not numb |

*The human subject was involved in physical activities that caused significant sweating between t = 150 min and 210 min, which could have had the effect of reducing the duration of the anesthesia effect because increased blood circulation may increase the speed of clearance of TC from the skin areas.

These results suggest that (1) storing the fabric loaded with 0.5 mg TC and 2 mg PVA per cm² at about 155° F. for 30 days does not destroy the anesthetizing ability, regardless of whether the loaded fabric was laminated to the 3M9832 tape before or after the high temperature storage. (2). The same high temperature storage seemed to significantly weaken the anesthetizing ability of the laminated sheet containing 0.3 mg TC and 2 mg PVA/cm², so the sheet (Sheet 1) only produced skin anesthesia that was delayed and of shorter duration. However, Sheet 1, with only 0.3 mg TC per cm², may have had weaker anesthetizing ability than the other sheets to start with.

### Example 33

### Separation of Heat Laminated Fabric-Polyurethane Film Laminate Caused by the Soaking of the Loading Solution

A fabric (Derma Sciences Dusoft 84148) was heat laminated to a polyurethane film (MedCo RTS 1716-11) with a heat press (Seiki Technologies, Type SK-HP3) with a heating temperature of 330° F. and a heating duration of 2 seconds. The heat lamination was done after placing the polyurethane film RTS 1716-11 and the Dusoft 84148 together between sheets of a plastic release liner (in this Example, 3M9956), with the plastic carrier layer of the polyurethane film facing toward the heating plate of the heat press. The bond was good and the two materials were not separable. However, after the fabric layer of the laminated sheet was dispensed with a blank loading solution (18.6% of a 25% PVA solution, 81.4% rubbing alcohol, 12 mL on 190 cm²) and the soaked laminate was allowed to dry at room temperature overnight, the fabric and the film became separable.

The heat lamination of the same fabric and film was repeated with the same 330° F. heating temperature but several different heating durations: 2 seconds, 3 seconds, 4 seconds, 5 seconds, and 6 seconds. After dispensing the same blank loading solution onto the fabric of each of the sheets (1.5 mL for each 28 cm² sheet) and drying the sheets in the 155° F. oven for one hour, all laminated sheets' film and fabric became separable.

These results suggest that the solvents in the loading solution can cause the separation of the film and fabric laminated together with 330° F. heating temperature and a heating duration as long as 6 seconds.

To explore whether higher heating temperature can produce a film-fabric laminate that can withstand the soaking of the loading solution without film damage, the following experiments were conducted: using the same method as described above, the RTS 1716-11 polyurethane film and the 3M9832F polyurethane film, respectively, were laminated to the Dusoft 84148 fabric using various heating temperatures and durations. The 3M 9832F film had no carrier. A loading solution of 1.3% tetracaine base (TC), 98.7% of the above blank loading solution, was made. The loading solution contained about 1.3% TC and 4.6% PVA. One and half mL of the loading solution was dispensed onto the fabric side of each of the laminated sheets (25 cm² each sheet). The solution soaked sheets were placed in a 155° F. oven for 60 min to evaporate off the solvents. The dried sheets were removed from the oven and allowed to cool to room temperature. Attempts were made to separate the film and the fabric of the dried sheets. The sheets were also examined for possible film damage. The heating temperature, heating duration, and test results are summarized in Table 2 below.

TABLE 2

| | Heating | Heating | Film separable | Film |
|---|---|---|---|---|
| Film | temp | duration | from fabric? | damage? |
| MedCo | 330° F. | 3 sec | Yes | No |
| RTS1716-11 | | | | |
| MedCo | 340° F. | 3 sec | Yes | No |
| RTS1716-11 | | | | |
| MedCo | 350° F. | 3 sec | Yes | No |
| RTS1716-11 | | | | |
| MedCo | 360° F. | 3 sec | Yes | No |
| RTS1716-11 | | | | |
| MedCo | 370° F. | 3 sec | Barely | No |
| RTS1716-11 | | | | |
| MedCo | 380° F. | 3 sec | No | No |
| RTS1716-11 | | | | |
| 3M9832F | 380° F. | 3 sec | Yes | Yes |

There results suggest that: (1) the Dusoft 84148 fabric and the MedCo RTS1716-11 film heat laminated together with a 380° F. heating temperature and a 3 second heating duration can withstand the soaking of the loading solution without separation. The film can withstand the heating temperature and duration without being damaged. (2) The 380° F. heating temperature and 3 second heating duration caused some film damage to the 3M9832F. The 3M9832F film did not have a carrier layer while the RTS 1716 film did have a thick plastic carrier layer that was in the path of heat transfer from the heating plate to the film. As a result, the 3M 9832F film might be exposed to higher temperature during the heat lamination process. It is possible that the damage to the 3M9832F film was caused by that higher temperature.

### Example 34

### Heat Laminating a Fabric and a Polyurethane Film with a 380° F. Heating Temperature and 3 Second Heating Duration and Effects of High Temperature Storage

In this Example, a fabric was heat laminated to a polyurethane film to form a laminate, and TC and PVA were impregnated into the fabric side of the laminate. The anesthetizing ability of the TC and PVA impregnated sheet was tested. The experimental procedures are as follows:

Step 1. A fabric (Derma Sciences Dusoft 84148) was heat laminated to a polyurethane film (MedCo RTS 1716-11) with a heat press (Seiki Technologies, Type SK-HP3) with heating temperature of 380° F. and heating duration of 3 seconds. The heat lamination method used was similar to that described previously.

Step 2. A TC loading solution with the following composition was made: 0.86% tetracaine base (TC), 99.14% of a blank solution (same as the blank loading solution in Example 33). This TC loading solution contained 0.86% TC and about 4.6% PVA, and had a density of about 0.91.

Step 3. Twelve mL of the TC loading solution made in Step 2 was evenly dispensed onto the fabric side of the laminated sheet (190 cm²) made in Step 1. The laminated sheet with the solution was placed into an oven with a temperature of about 155° F. for 60 min to evaporate off the solvents. The dried sheet was removed from oven and allowed to cool to room temperature. There was no damage to the film part of the laminated sheet, and the fabric and film were not separable. This dried sheet contained about 0.5 mg TC and 2.7 mg PVA per cm².

Step 4. The sheet made in Step 4 was cut in half. One of the halves was stored at room temperature and the other half was placed into an oven at a temperature of about 155° F. for the next 12 days.

Step 5. A 1 cm×2 cm piece of the room temperature half sheet made in Step 4 was cut out from the larger sheet and placed onto the forearm skin area of a human subject which had already been covered with fine water droplets sprayed on with a spray bottle. The fabric side of the sheet was in contact with the skin. After 60 min, the sheet piece was removed from the skin. The skin area under the sheet was deeply numb as determined by poking with an end of a straightened paper clip.

Step 6. After 12 days of storage in the 155° F. oven, the half sheet made and placed in the oven in Step 4 was removed from the oven and allowed to cool to room temperature. Four days later, the anesthetizing ability of the sheet was tested with a method similar to that used in Step 5. The treated skin area was numb when tested at t=60 min (60 min from the beginning of the sheet application) and t=240 min.

These results show that after 12 days of storage at about 155° F., the laminated sheet does not lose its anesthetizing ability. There was no lamination adhesive in the sheet in this Example, so that potential interaction between the drug and the lamination adhesive was avoided.

### Example 35

### Sheets with Ahlstrom Fabric, with No Fabric, and with Add-on Barrier Film

Step 1. A tetracaine (TC) loading solution with the following composition was made:

2% TC, 32.7% of a 25% PVA:75% water solution, and 65.3% rubbing alcohol.

Step 2. 2.6 mL of the TC loading solution in Step 1 was evenly dispensed onto a polyurethane film with paper carrier (80 cm², 3M9832F). The film covered with the solution was placed into an oven with a temperature of about 155° F. for 30 min to evaporate off the solvents. This process coated the 3M9832F film with about 0.6 mg TC and 2.5 mg PVA per cm², and caused the film to wrinkle a little on the paper carrier.

Step 3. 3 mL of the TC loading solution in Step 1 was dispensed evenly onto the fabric side of a 55 cm² pre-heat laminated sheet [Ahlstrom SX567 polyester fabric heat laminated to MedCo RTS1716-11 polyurethane film (with plastic carrier). Heating temperature was 380 F, heating duration was 3 seconds, and the lamination method was similar to that described previously]. The solution loaded sheet was dried in the same oven as in Step 2 for 60 min to evaporate off the solvents. The resulting dried laminated sheet had about 1 mg TC and 4.1 mg PVA per cm².

Step 4. The Ahlstrom SX567 polyester fabric was loaded with 3 mg TC and 3 mg PVA per cm². The loading method used was similar to that described previously.

The following skin tests were conducted:

### Test 1

Fine water droplets were sprayed onto the skin of the back side of a human subject's hand. A piece of the polyester fabric loaded with 3 mg TC and 3 mg PVA per cm² made in Step 4 (1 cm×2 cm, no barrier film) was placed on the wet skin. The fabric was then covered with a piece of a plastic film (about 3 cm×4 cm, “Glad” brand Cling Wrap, a common kitchen item).

### Test 2

Fine water droplets were sprayed onto the forearm skin of a human subject. A piece, 1 cm×2 cm, of the 3M9832F film coated with 0.6 mg TC and 2.5 mg PVA per cm², made in Step 2, was placed on the wet skin, with the side with TC and PVA coating in contact with the skin.

### Test 3

Fine water droplets were sprayed onto the forearm skin of a human subject. A piece, 1 cm×2 cm, of the laminated sheet made in Step 3, containing about 1 mg TC and 4.1 mg PVA per cm², was placed on the wet skin.

After 60 min (t=60 min), all three sheets were removed from the skin areas. It was observed that the skin area in Test 1 was still wet, but that in Tests 2 and 3 the skin under the sheets was dry. This likely was due to the lower MVTR of the plastic film in Test 1 than the barrier films in Tests 2 and 3. It was also observed that some shiny residue was left on the skin in Test 2, likely because the TC and PVA on the barrier film were transferred to the skin during the application. All three skin areas were deeply numb.

At t=3 hours, all three skin areas were deeply numb.

At t=5 hours, all three skin areas were numb.

At t=5.5 hours, all three skin areas were numb, but the profoundness of the numbing is decreasing in all three skin areas.

At t=6 hours, all three skin areas were slightly numb, but most of the skin anesthesia was gone.

These results suggest that: (1) a fabric coated with TC and PVA can be an independent product, which can be used with a commonly available plastic film (a common kitchen item in this case) and water to produce skin anesthesia. (2) TC and PVA can be coated to a barrier film without a fabric layer, and such a coated barrier film can produce good skin anesthesia. However, it may leave TC/PVA residual on the skin because the TC and PVA were not “fastened” to the sheet, as in sheets in which the TC and PVA are impregnated in the fabric layer. (3) The Ahlstrom SX567 polyester fabric can be a good fabric layer in the fabric-barrier film laminated sheet.

### Example 36

A viscous aqueous solution (1.6% carbopol 981 NF, 0.9% sodium hydroxide, 97.5% water) was made and placed into a plastic squeeze bottle with a long dispensing nozzle (½ oz. volume oval plastic bottle with “Yorker spout” cap, purchased from Industrial Container and Supply Co., Utah). A drop of the viscous solution was squeezed out of the bottle onto the skin of the back side of a human subject's hand. The drop of the viscous solution was spread into a thin layer with the long nozzle of the squeeze bottle to cover an area slightly larger than 1 cm×2 cm. A 1 cm×2 cm piece was cut from a previously made TC and PVA impregnated laminated sheet (0.5 mg TC and 2.7 mg PVA per cm² dried on the Dusoft 84148 fabric pre-heat laminated on the MedCo RTS 1716-11 polyurethane film) and placed on the wet skin. After 60 min, the sheet piece was removed from the skin. The skin was deeply numb and dry. The sheet piece adhered very well to the skin during the entire 60 min application time.

In applications in which the liquid (in the Sheet Liquid Combination System) has to be placed precisely (e.g. close to eyes), spraying the liquid on the skin may not be adequate because it can be difficult to aim the liquid when spraying. The sprayed liquid may also run because liquid that can be sprayed typically must have low viscosity. In those applications, as shown in this Example, viscous liquid can be applied with a squeeze bottle or other convenient (may be disposable) container with an applicator (e.g. the long nozzle in this case). The sheet can then be applied over the liquid layer.

### Example 37

The following experiment was conducted to demonstrate the effect of barrier film (MVTR control layer) on a sheet's ability to produce skin anesthesia.

Step 1. A tetracaine (TC) loading solution with the following composition was made: 0.7% tetracaine base, 11.2% of a 25% PVA:75% water solution, 88.1% rubbing alcohol. The density of the solution was about 0.91.

Step 2. Fifteen mL of the TC loading solution made in Step 1 was evenly dispensed onto a piece of fabric (190 cm², single ply, Dusoft 84148) resting on a release liner. The solution soaked fabric was placed into an oven with a temperature of about 155° F. for 40 minutes to evaporate off the solvents. The dried fabric had about 0.5 mg TC and 2 mg PVA per cm².

Step 3. A drop of a viscous aqueous solution (1.6% Carbopol 981 NF, 0.9% sodium hydroxide, 97.5% water) was squeezed out of a squeeze bottle and spread, with the help of the long nozzle of the squeeze bottle, onto a forearm skin area (slightly larger than 1 cm×2 cm) of a human subject. A 1 cm×2 cm piece was cut from the dried fabric produced in Step 2 (loaded with 0.5 mg TC and 2 mg PVA per cm²) and placed on the wet skin.

Step 4. In a separate skin area of the same forearm, the procedure in Step 3 was repeated, except that the dried fabric was laminated with the 3M9832 polyurethane tape, using the tape's adhesive, just before the test.

### Observations:

The piece of the fabric in Step 3 became visibly wet right after it was placed on the wet skin. The viscous solution easily penetrated the fabric layer, and could be removed (partially) if touched by a finger (the solution was not touched during this test so it was not removed). The fabric in Step 3 became visibly dry at t=10-15 min (t=0 was when the sheet was applied) and stayed on the skin very well until it was removed from the skin at t=60 min. The skin under the fabric in Step 3 was not numb at all at t=60 min, and not numb at all in the next 6 hours.

At t=60 min, the laminated sheet in Step 4 was removed and the skin treated by it was deeply numb. The skin area was dry but the fabric part of the sheet piece was still a little damp.

In the above experiment, water was present for about 10 minutes on the skin area under the fabric without a barrier film (by visual observation of dryness), and for about 60 minutes under the laminated sheet. It is a little surprising that this difference in the water presence time produced such a dramatic difference in skin anesthesia: no anesthesia at all vs. deep anesthesia.

The above results reveal the importance of keeping water on the skin for a long enough time to obtain the anesthetic effect with tetracaine. Keeping water for 10 minutes or less (as measured by visual observation of dryness) may be insufficient for obtaining skin anesthesia, at least in some individuals and under some conditions.

It should be pointed out that barrier film-fabric laminate may not be the only configuration in the sheet and liquid combination system of the current invention that can keep water on the skin for a long enough time to produce skin anesthesia. It is possible that a fabric with low enough MVTR, without a barrier film, may also be able to keep water on the skin for a sufficient period of time. It is also possible that a barrier film impregnated with tetracaine, without a fabric layer, can keep the water on the skin surface for long enough time (Example 38).

### Example 38

The following attempt was made to impregnate TC into a polyurethane film, so the TC impregnated film alone could function as the sheet in the Sheet Liquid Combination system of the current invention.

One-tenth of a gram of tetracaine base (TC) was placed into a small glass vial. A 2 cm×8 cm piece of a polyurethane film (MedCo RTS 1717-11, plastic carrier removed) was also placed into the glass vial. One and six-tenths grams of rubbing alcohol was then added into the glass vial. After gentle shaking and waiting, all TC particles dissolved in the rubbing alcohol. The entire polyurethane film was submerged in the solution.

The above system was let sit in room temperature for 48 hours before the polyurethane film was retrieved from the solution. The retrieved film was rinsed with water, dried with a Kleenex paper tissue, and placed into an oven with temperatures of about 155° F. for 30 minutes to evaporate off any solvent still in the film.

A thin layer of a viscous aqueous solution (1.6% Carbopol 981 NF, 0.9% sodium hydroxide, 97.5% water) was spread on the back side of a human subject's hand. A 1 cm×2 cm piece of the dried film was applied onto the wet skin. After a 60 min application period (during which the film stayed adhered to the skin surface very well), the film was removed from the skin. The skin area treated by the film was deeply numb. The skin surface was still wet when the film was removed, which was probably due to the fact that this film is thicker (thus probably had lower MVTR) than some other barrier films used in previous tests (e.g. 3M9832F, MedCo RTS1716-11).

The above experiment reveals that a polyurethane film can absorb enough TC (when submerged in a TC solution) and can release TC at fast enough rate (when in contact with the appropriate vehicle solution) to anesthetize the skin within 60 min. Other barrier films, especially the ones of absorbent materials such as silicone and latex, may be able to do the same.

In this case, the sheet in the Sheet and Liquid Combination System was the film alone, impregnated with tetracaine. The adhesion agent (Carbopol 981 NF, pH neutralized with sodium hydroxide) was in the vehicle liquid. No fabric layer was used. Alternatively, an adhesion agent, such as PVP or PVA, can be placed in the drug solution and impregnated into the film via the same diffusion process for impregnating the drug (TC in this case) into the film.

### Example 39

### Skin Anesthesia Test for Sheets that Experienced Long Time High Temperature Storage, were Made with Various Lamination Adhesives, were Heat Laminated, Etc.

The following samples were made for skin anesthesia tests:

### Sample 1

0.5 mg TC (tetracaine) and 2 mg PVA (polyvinyl alcohol) per cm² was impregnated into the Dusoft 84148 fabric by a process as previously described. The loaded fabric (dried) was laminated to the 3M 9832 polyurethane tape using the tape's adhesive. The laminated sheet was stored in an oven with temperatures cycling between about 62° C. and about 68° C. for 43 days. The fabric part (impregnated with TC and PVA) of the sheet became slightly yellow due to the long storage time at such a high temperature.

### Sample 2

0.3 mg TC and 2 mg PVA per cm² was impregnated into the Dusoft 84148 fabric by a process as previously described. The loaded fabric (dried) was laminated to the 3M 9832F polyurethane film using 3M 1504 XL transfer adhesive. The laminated sheet was stored at room temperature for 37 days before the test.

### Sample 3

Same was produced and stored under conditions similar to the sheet in Sample 2, except the 3M1524 transfer adhesive, instead of the 3M1504 XL transfer adhesive, was used.

### Sample 4

The Dusoft 84148 fabric was laminated to the MedCo RTS 1716-11 polyurethane film by heat using a heat lamination process similar to that described previously (heating temperature of 380° F., heating duration of 3 seconds). 0.5 mg TC and 2 mg PVA per cm² was impregnated into the fabric of the laminated sheet using a method described previously. The laminated sheet impregnated with the TC and PVA was stored in an oven with temperatures cycling between about 62° C. and about 68° C. for 12 days before the test.

### Sample 5

0.5 mg TC and 2 mg PVA per cm² was impregnated into the Dusoft 84148 fabric by a process as previously described. The TC and PVA impregnated fabric (dried) was laminated the 3M9832F polyurethane film with a heat lamination process similar to that described previously. The heating temperature and duration was 330° F. and 2 seconds, respectively. The TC and PVA impregnated laminated sheet was stored in an oven with temperatures cycling between about 62° C. and about 68° C. for 29 days before the test.

### Sample 6

The Dusoft 84148 fabric was laminated to the 3M9832F polyurethane film by heat using a heat lamination process similar to that described previously. The heating temperature and duration was 330° F. and 2 seconds, respectively. 0.3 mg TC and 2 mg PVA per cm² was then impregnated into the fabric of the laminated sheet using a process similar to that previously described. The TC and PVA impregnated laminated sheet was stored in an oven with temperatures cycling between about 62° C. and about 68° C. for 33 days before the test.

The following skin tests were conducted: fine water droplets were sprayed onto the skin of the back of a human subject's hand with a spray bottle. Each of the six sheets, about 1 cm×2 cm, was placed on the wet skin and removed after 60 minutes. All six skin areas were dry when the sheets were removed. The numbness of each of the skin areas treated by the sheets was tested by poking the area with a straightened paper clip. The results are summarized in Table 3 below.

TABLE 3

| Test | Sample | | | Sample | | Sample |
|---|---|---|---|---|---|---|
| Time* | 1 | Sample 2 | Sample 3 | 4 | Sample 5 | 6 |
| 60 min | deeply | deeply | Not | deeply | Not | Not |
| | numb | numb | numb | numb | numb | numb |
| 120 min | deeply | deeply | deeply | deeply | deeply | Not |
| | numb | numb | numb | numb | numb | numb |
| 240 min | numb | numb | Not | numb | Not | Not |
| | | | numb | | numb | numb |

*Test time = 0 when the application of the sheets started

These results suggest that:

(1) The sheet with the configuration and formulation exemplified by Sample 1 has very stable anesthetizing ability. Storing the sample at about 62° C. and 68° C. for 43 days accelerated the aging of the sample so much that the fabric started to yellow, yet the anesthetizing ability of the sheet was not detectably compromised. If one uses the rule of thumb that for every 10° C. temperature increase, the rate of chemical or physical process that can (eventually) compromise the drug performance is increased by a factor of 3, the storage conditions of about 62° C. to about 68° C. for 43 days is equivalent to storage at 25° C. for approximately 10 years.

(2) Sample 2, the sheet with the 3M 1504XL transfer adhesive as the lamination adhesive, did not lose the anesthetizing ability after 37 days storage at room temperature. However, in a separate test, after the same sheet was stored at about 62° C. to about 68° C. for 29 days, it did not produce numbness in a similar test. Without being limited to any one theory, it is possible that the 0.3 mg/cm² TC quantity of the active ingredient is sufficient to provide only borderline effectiveness such that such sheets sometimes produce the desired numbness and sometimes do not. It is also possible that the 3 m1504XL transfer adhesive slowly destroys the anesthetizing ability, so that the anesthetizing ability of the sheet was destroyed after 29 days at about 62° C. to about 68° C. but not lost after 37 days at about 25° C.

(3) Sample 3, the sheet laminated with the 3M 1524 transfer adhesive, had reduced anesthetizing ability (not numb at t=60 min but numb at t=120 min) after 37 days storage at room temperature. In a separate test, the same sheet that was stored at a temperature of about 62° C. to about 68° C. for 29 days, and when tested under similar conditions to those spelled out above, did not produce numbness. Again, without being limited to any one theory, it is possible that the 0.3 mg/cm² TC quantity is of a dosage of active ingredient so as to be borderline effective such that such sheets sometimes produce numbness and sometimes do not. It is also possible that the 3M1524 transfer adhesive slowly destroys anesthetizing ability, so that the anesthetizing ability of the sheet was partially destroyed after 37 days at 25° C. If the latter is the case, the 3M1524 transfer adhesive probably destroys the anesthetizing ability faster than the 3M1504 XL adhesive.

(4) Sample 4, a pre-heat laminated sheet comprising the film (MedCo RTS 1716-11 polyurethane) and the fabric (Derma Sciences Dusoft 84148 rayon-polyester blend) with TC and PVA impregnated in the fabric part, did not lose its anesthetizing ability after 12 days of storage at a temperature of about 62° C. to about 68° C. Because the sheet does not contain lamination adhesive, its anesthetizing ability is expected to be very stable over long term storage because potential adverse interactions between the drug formulation and the lamination adhesive are avoided.

(5) For Sample 5, heating the TC and PVA impregnated fabric to 330° F. for 2 seconds in the heat lamination process and storing the laminated sheet at a temperature of about 62° C. to about 68° C. for 29 days significantly reduced, but did not completely destroy, the anesthetizing ability of the sheet.

For sample 6, the pre-heat laminated sheet impregnated with 0.3 mg TC and 2 mg PVA per cm² lost all its anesthetizing ability after 33 days of storage at a temperature of about 62° C. to about 68° C. Again, it is possible that the 0.3 mg/cm² TC quantity is insufficient to be consistently effective such that such sheets sometimes produce numbness and sometimes do not. It is also possible that the 33 day high temperature storage reduced the originally already marginal anesthetizing ability to the level that it cannot produce skin anesthesia.

In this and other Examples, when a sheet that was stored at elevated temperatures of at least 45 C before testing for its anesthetizing ability, the duration of storage of the sheet at room temperature is typically not mentioned. That is because the physical or chemical process that reduces or destroys anesthetizing ability usually takes place at a much faster rate at the elevated temperatures than at room temperature, such that the duration of room temperature storage is insignificant.

### Example 40

The following samples were made for skin anesthesia tests:

Step 1. A blank loading solution was made by mixing 18.6 parts of a 25% PVA:75% water solution with 81.4 parts of rubbing alcohol. The density of this blank loading solution was about 0.91.

Step 2. TC Loading Solution A was made by dissolving 1.04% of tetracaine base (TC) in 98.96% of the blank loading solution made in Step 1. Twelve mL of the TC Loading Solution A was dispensed onto a fabric (190 cm², Dusoft 84148 rayon-polyester blend fabric from Derma Sciences) resting on a release liner.

Step 3. TC Loading Solution B was made by mixing the TC Loading Solution A made in Step 2 with an equal weight of the blank loading solution made in Step 1. Twelve mL of the TC Loading Solution B was dispensed onto a 190 cm² of the same fabric as in Step 2 resting on a release liner.

Step 4. TC Loading Solution C was made by mixing the TC Loading Solution B made in Step 3 with an equal weight of the blank loading solution made in Step 1. Twelve mL of the TC Loading Solution C was dispensed onto a 190 cm² of the same fabric as in Step 2 resting on a release liner.

Step 5. The three solution soaked fabric sheets made in Steps 2-4 were dried in an oven at a temperature of about 155° F. for 60 min. The dried fabric sheets contained 0.6 mg TC+2.7 mg PVA/cm², 0.3 mg TC+2.7 mg PVA/cm², and 0.15 mg TC+2.7 mg PVA/cm², respectively.

Step 6. Each of the TC+PVA impregnated fabric pieces made in Step 5 was laminated to the 3M9832 polyurethane tape using the tape's adhesive.

Step 7. The following skin anesthesia tests were conducted immediately after Step 6: fine water droplets were sprayed onto the skin of the back side of a human subject's hand. A piece, about 1 cm×2 cm, was cut from each of the laminated sheets made in Step 6 and placed on the wet skin. After 45 minutes (t=45 min), each sheet was lifted and the skin area was scratched with the end of a straightened paper clip to test the degree of numbness (anesthesia) induced by the sheet. The sheets were then replaced on the original skin area and kept there until t=60 min, when all three sheets were removed. The degree of skin anesthesia in the three skin areas treated by the sheets was tested with the same straightened paper clip at several later time points. The skin numbness results are summarized in Table 4 below.

TABLE 4

| Time (from | | | |
|---|---|---|---|
| the start of | | | |
| application) | 0.15 mg TC/cm² | 0.3 mg TC/cm² | 0.6 mg TC/cm² |
| 45 min | Not numb | Numb in most of | Numb in entire |
| | | the area, but not | area |
| | | entire area. | |
| 60 min | Not numb | Numb | Numb |
| 120 min | Numb | Numb | Numb |
| 300 min | Numb | Numb | Numb |

### Remarks:

In this polyurethane film-rayon/polyester blend laminated sheet, 0.15 mg TC/cm² produced delayed anesthesia effect (compared with the sheet containing 0.6 mg TC/cm²). 0.3 mg TC/cm² was better, but still not as good as 0.6 mg TC/cm². Because it is believed that different individuals can have 3-4 fold difference in skin permeability, and different skin conditions (cold vs. warm, hydrated vs. dry) can also cause difference in skin permeability, 0.6 mg TC/cm² or higher TC quantity per cm² should be used in a product so that the product can produce the desired effect in most users.

With a “lighter sheet”, such as a polyurethane film impregnated with TC without a fabric layer, the amount of TC per cm² needed to produce a maximum degree of skin anesthesia can be lower, because a lower amount of TC may be held by such a ‘lighter” sheet than the sheets used in the above tests.

### Example 41

With methods similar to that described previously, 3 Dusoft 84148 fabric sheets were loaded with the following amounts of PVA, respectively:
Fabric sheet A: 2 mg PVA/cm²
Fabric sheet B: 6.2 mg PVA/cm²
Fabric sheet C: 10 mg PVA/cm²

Each of the PVA-loaded fabric sheets (after drying) was laminated with the 3M 9834 (polyurethane) tape, using the tape's adhesive. The fabric's side that faced up during the drying process was the side that adhered to the tape's adhesive layer.

A 2.5 cm×4 cm piece was die cut from each of the laminated sheets above. Water was sprayed on the forearm skin of a human subject to form densely populated water beads on the skin. Each of the 2.5 cm×4 cm sheets was applied on the wet skin, and the sheets and surrounding skin areas were gently tapped with a Kleenex tissue to ensure good contact and remove excess water on the skin. The human subject did routine lab work in the next two hours of test period so the skin areas were stretched and bent accordingly. The sheets' adhesion to the skin was observed for the two 2 hour test periods, with the following results:

Right after the sheets were applied (t=0): all sheets adhered to skin well.

At t=20 min: the 2 mg PVA/cm² sheet had wrinkles and was partially separated from the skin. The 6.2 mg PVA/cm² and 10 mg PVA/cm² sheets stayed adhered well to the skin.

At t=70 min: the 2 mg PVA/cm² sheet had wrinkles and was about 40% separated from the skin. The 6.2 mg PVA/cm² and 10 mg PVA/cm² sheets stayed adhered well to the skin.

At t=120 min: all three sheets were removed from the skin. The 2 mg PVA/cm² sheet had wrinkles and was about 50% separated from the skin just before removal. The part that was still adhered to the skin had post-it sticker kind of adhesion strength. The 6.2 mg PVA/cm² and 10 mg PVA/cm² sheets stayed adhered to the skin well until their removal. Peeling those two sheets off the skin lifted the skin slightly. Their adhesion strength was much stronger than that of the 2 mg PVA/cm² sheet. The adhesion strength of the 10 mg PVA/cm² sheet was not much stronger than the 6.2 mg PVA/cm² sheet.

These results suggest that amounts of PVA higher than 2 mg/cm² or about 6 mg/cm² may make the sheet to have stronger adhesion to the skin. However, for applications in which the skin area is not expected to be bent or stretched, such as facial skin anesthesia before painful procedures, strong adhesion may be unnecessary and 2 mg PVA/cm² may provide strong enough adhesion.

In another experiment, 10 mg PVA/cm² and 0.5 mg tetracaine/cm² was loaded to a Dusoft 84148 fabric with a method similar to that described previously. The PVA and tetracaine-loaded fabric sheet was laminated with the 3M 9832 tape, using the tape's adhesive. The fabric's side that faced up during the drying process was the side that adhered to the tape's adhesive layer. A 1 cm×2 cm piece was cut from the laminated sheet. Water was sprayed on the forearm skin of a human subject to form densely populated water beads on the skin. The 1 cm×2 cm sheet was applied on the wet skin, and the sheet and surrounding skin area were gently tapped with a Kleenex tissue to ensure good contact and remove excess water on the skin. The sheet adhered to the skin very well during the entire 45 min test period. At 45 min from the application, the sheet was removed from the skin. Peeling the sheet off the skin lifted the skin slightly. The skin was deeply numb and dry when the sheet was removed. No visible residue was left on the skin. These results suggest that the laminated sheet with the 10 mg PVA and 0.5 mg tetracaine/cm² formulation can successfully anesthetize the skin.

### Example 42

Local anesthetic agent lidocaine and anti-infection agent chlorhexidine are both loaded into a polyurethane film using the method similar to that in Example 38. When this lidocaine and chlorhexidine loaded film is applied to cover a wound surface, such as a fresh and severe burn wound surface, the bodily fluid from the wound surface would contact the film and the drugs lidocaine and chlorhexidine can be released from the film using the bodily fluid as the diffusion vehicle. This approach can achieve several benefits: minimize the pain (lidocaine's function), reduce the infection potential (chlorhexidine's function), and isolate the wound surface from the external environment (the film's function) which can further reduce the potential of infection. This approach can be very useful in emergency situations, such as war act-caused injuries, where the thorough treatment of the wound cannot be performed immediately and reducing the infection potential and pain for a few hours before the thorough treatment with a very simple method is very important.

In this Example, the “liquid” in the “sheet and liquid combination” system of the current invention is the bodily fluid oozing out of the wound. If that's not enough fluid, a water containing fluid can be sprayed on the wound surface or film as additional “fluid”.

A polyurethane film is particularly suitable for this purpose. A polyurethane film with proper thickness, such as 1/1000 inch or 1/2000 inch, can be a barrier to viruses and bacteria while “breathable” to water vapor so that the wound surface is not completely occluded which can mean more comfort to the patient. More important, as shown in Example 38, a polyurethane film can absorb sufficient drug and release it at sufficient rate to achieve a therapeutic effect. While the drug in Example 38 is a local anesthetic, the polyurethane film should be able to absorb and release many other drugs with sufficient amounts and rates.

It will be appreciated by those having skill in the art that many changes may be made to the details of the above-described embodiments without departing from the underlying principles of the invention. The scope of the present invention should, therefore, be determined only by the following claims.

1. A system for delivering tetracaine into human skin, comprising:
a sheet of a material impregnated with tetracaine and free of water; and
a vehicle liquid comprising water;
wherein said sheet and said vehicle liquid are stored separately, wherein said sheet and vehicle liquid are joined prior to or during an application on human skin, and wherein said sheet has sufficiently low MVTR for tetracaine delivery, wherein application of said sheet on a normal human skin surface with said vehicle liquid placed between said sheet and said normal human skin surface is sufficient to achieve anesthesia in said normal human skin within 120 minutes of application under normal ambient conditions.

2. A system for delivering tetracaine into human skin, comprising:
a sheet of a material impregnated with a pH modifying agent;
a vehicle liquid comprising tetracaine and water and with pH lower than about 6; and
an adhesion agent in either said sheet or said vehicle liquid;
wherein said sheet and said vehicle liquid are stored separately, wherein said sheet and vehicle liquid are joined prior to or during an application on human skin, wherein said sheet has sufficiently low MVTR for tetracaine delivery, and wherein the application of said sheet to a normal human skin surface with an appropriate quantity of said vehicle liquid placed between said sheet and said normal skin surface is sufficient to produce anesthesia in said normal human skin within 120 minutes under normal ambient conditions, and wherein said pH modifying agent impregnated in said sheet is sufficient to increase the pH of said vehicle liquid applied on said human skin to higher than about 6.5.

3. (canceled)

4. The system of claim 1, wherein said vehicle liquid or said sheet comprises an adhesion agent.

5-6. (canceled)

7. The system of claim 1, wherein the quantity of said vehicle liquid applied between said sheet and said skin is in the range of between about 2 to about 200 mg/cm².

8-10. (canceled)

11. The system of claim 1, wherein said sheet comprises an MVTR control layer whose MVTR is below 2,000 g/m²/24 hours.

12-14. (canceled)

15. The system of claim 4, wherein said adhesion agent is selected from the group of polyvinylpyrrolidone, polyvinyl alcohol, polyacrylic polymers, xantham gum, gum Arabic, poly ethylene glycol, glycerin, or a combination thereof.

16-19. (canceled)

20. The system of claim 1, wherein said sheet further comprises a fastening agent for fastening said tetracaine on said sheet.

21. (canceled)

22. The system of claim 1, wherein said sheet comprises a polyurethane film.

23-25. (canceled)

26. The system of claim 1, wherein said sheet has a dry-ending MVTR.

27-35. (canceled)

36. The system of claim 1, wherein the application of said sheet applied on normal human skin with an appropriate quantity of said vehicle liquid placed between said sheet and a normal human skin area is sufficient to achieve anesthesia in said normal human skin within 60 minutes under normal ambient conditions.

37. (canceled)

38. The system of claim 1, wherein at least one part of said sheet comprises at least 0.1 mg tetracaine per cm².

39-45. (canceled)

46. The system in claim 1, wherein at least one part of said sheet comprises at least 1 mg PVA per cm².

47. (canceled)

48. The system of claim 1, wherein said vehicle liquid is contained in a spray bottle.

49-51. (canceled)

52. The system of claim 1, wherein said sheet comprises a layer of adhesive comprising tetracaine that is adhesive to intact human skin without a vehicle liquid, is not capable of delivering tetracaine at a sufficient rate to numb normal human skin within 120 minutes under normal ambient conditions without a vehicle liquid, and is capable of delivering tetracaine at a sufficient rate to numb normal human skin with said vehicle liquid within 120 minutes under normal ambient conditions.

53-54. (canceled)

55. The system of claim 1, wherein the sheet comprises a barrier film layer and a fabric layer.

56-61. (canceled)

62. A method for reducing pain associated with herpes zoster, comprising:
placing a vehicle liquid comprising water and a sheet of a material impregnated with tetracaine on a human skin area suffering from the pain associated with herpes zoster in acute eruptive phase or post herpetic neuralgia, wherein said vehicle liquid is placed between said human skin area and said sheet; and
maintaining said sheet on said human skin area for a sufficient period of time to achieve reduction of said pain, wherein said sheet is constructed to have an low enough MVTR to keep said water between the skin and the sheet for a sufficient period of time to achieve pain reduction within 120 minutes for patients with normal skin temperatures and under normal ambient conditions.

63-64. (canceled)

65. A method for reducing musculoskeletal pain, comprising:
placing a vehicle liquid comprising water and a sheet of a material impregnated with tetracaine on a target human skin area under which musculoskeletal pain or a trigger point associated with musculoskeletal pain exists, wherein said vehicle liquid is placed between said human skin area and said sheet; and
maintaining said sheet on said human skin area for at least 30 minutes, wherein said sheet's MVTR is lower than 5000 g/m²/24 hour.

66. (canceled)

67. A method for reducing neuropathic pain, comprising:
placing a vehicle liquid comprising water and a sheet of a material impregnated with tetracaine on a human skin area in which or under which neuropathic pain exists, wherein said vehicle liquid is placed between said human skin area and said sheet; and
maintaining said sheet on said human skin area for at least 30 minutes, wherein said sheet's MVTR is lower than 5000 g/m²/24 hour.

68-94. (canceled)

95. The method of claim 65, wherein said method further comprises the step of applying local heat to said sheet already applied on the skin.

96-114. (canceled)

115. The method of claim 62, wherein the application of said sheet applied on normal human skin with a quantity of said vehicle liquid placed between said sheet and the normal human skin area is sufficient to achieve anesthesia in said normal human skin within 60 minutes under normal ambient conditions, and wherein the quantity of said vehicle liquid is in the range of 5 to 200 mg per cm².

116-122. (canceled)

123. A system for delivering tetracaine into human skin, comprising:
a sheet comprising tetracaine and being free of water; and
a vehicle liquid comprising water;
wherein the application of said sheet applied on said skin with a quantity of 25 mg/cm² of said vehicle liquid in between the sheet and the skin is sufficient to achieve anesthesia in normal human skin within 60 minutes under normal ambient conditions, and wherein said application of said sheet and said vehicle liquid is capable of providing proper adhesion between the sheet and the normal human skin surface.

124-138. (canceled)

139. A system for delivering a drug into human skin, comprising a first component and a second component, wherein
the first component comprises a sheet of a material impregnated with a drug and is free of any substance incompatible with the drug, and
the second component comprises a substance incompatible with said drug,
wherein the first or second component alone, when applied on normal human skin, cannot deliver the drug at sufficient rates to achieve the desired clinical effect, wherein the application of the first component being placed on human skin with the second component being placed between the first component and the human skin can deliver the drug at sufficient rates to achieve the desired clinical effect.

140. The system of claim 139, wherein said drug is selected from the group of ketamine, gabapentin, tetracaine or benzocaine or other ester type local anesthetics, ketoprofen or diclofenac or other nonsteroidal anti-inflammatory drugs, capsaicin, and N-methyl-D-asparate (NMDA) receptor antagonists.

141-148. (canceled)

149. A system for providing analgesia to a skin or wound surface with a damaged or no stratum corneum layer, comprising a sheet impregnated with local anesthetic-ion exchange resin complex.

150-151. (canceled)

152. A system for transdermal delivery of diclofenac, comprising
a solution comprising diclofenac,
a sheet comprising a fluid retention layer and a barrier film, and
an adhesion agent in said solution or impregnated in said sheet, wherein the application of said solution and said sheet, with said solution being in between said sheet and a human skin surface, being sufficient to properly adhere said sheet to a normal human skin surface for at least 15 minutes under normal ambient conditions to allow delivery of the diclofenac.

153. A film for treating a wound, comprising a barrier film with MVTR lower than 5,000 g/m²/24 hours and a drug selected from the group of local anesthetics and anti-infection agents, wherein said drug is impregnated in said film.

154-155. (canceled)

156. A sheet for delivering tetracaine into human skin, comprising at least 0.1 mg tetracaine/cm², wherein said sheet is free of water and said sheet's MVTR is lower than 2,000 g/m²/24 hours.

157-183. (canceled)

184. The method of claim 65, wherein the application of said sheet applied on normal human skin with a quantity of said vehicle liquid placed between said sheet and the normal human skin area is sufficient to achieve anesthesia in said normal human skin within 60 minutes under normal ambient conditions, and wherein the quantity of said vehicle liquid is in the range of 5 to 200 mg per cm².

185. The method of claim 67, wherein the application of said sheet applied on normal human skin with a quantity of said vehicle liquid placed between said sheet and the normal human skin area is sufficient to achieve anesthesia in said normal human skin within 60 minutes under normal ambient conditions, and wherein the quantity of said vehicle liquid is in the range of 5 to 200 mg per cm².
